(19) United States
(12) Patent Application Publication
(10) Pub. No.: US 2026/0283479 A1
(43) Pub. Date: Sep. 24, 2026

(54) RESPIRATORY CYCLE ONSET DETECTION

(71) Applicant: Whoop, Inc., Boston, MA (US)

(72) Inventor: Rahul Kumar Krishnaiah Sevakula, Chestnut Hill, MA (US)

(21) Appl. No.: 19/083,548

(22) Filed: Mar. 19, 2025

(51) Int. Cl. A61B 5/0205; A61B 5/00; A61B 5/024; A61B 5/08; A61B 5/113; G16H 10/60; G16H 40/67

(52) U.S. Cl. CPC A61B 5/0205; A61B 5/4812; A61B 5/681; A61B 5/7221; A61B 5/725; A61B 5/7267; A61B 5/7278; G16H 10/60; G16H 40/67; A61B 5/02405; A61B 5/02416; A61B 5/02438; A61B 5/0816; A61B 5/113; A61B 2562/0219

(57) ABSTRACT

Pulse data including a plurality of heart pulse samples of a user during a time window may be received from a wearable physiological monitor worn by the user. The pulse data may be mapped into a feature space that encodes individual heart pulse samples according to respiratory cycle stage. A first estimated respiratory waveform of the user during the time window may be generated from the pulse data mapped to the feature space and a plurality of respiratory onsets for the user during the time window calculated based on a plurality of local extrema of the first estimated respiratory waveform. Motion data may also be used, either alone or in a multi-model system, to evaluate user respiration.

### TECHNICAL FIELD

The present application relates to wearable physiological monitoring systems, and more particularly to detecting and applying respiratory variability.

### BACKGROUND

Physiological monitoring systems can monitor heart rate activity via sensors such as photoplethysmography (PPG) sensors or electrocardiogram (ECG) sensors, and use this data to provide metrics for sleep performance, activity, strain, recovery, and so forth. While other metrics can be derived from such sensor data, delineating individual respiratory cycles by identifying the start of inspiration may be useful in more accurately evaluating physiological conditions associated with the user such as recovery rate, sleep stage, instantaneous respiratory rate, and so forth.

There remains a need for improved and convenient methods for respiratory cycle onset determination and physiological condition detection.

### SUMMARY

Pulse data including a plurality of heart pulse samples of a user during a time window may be received from a wearable physiological monitor worn by the user. The pulse data may be mapped into a feature space that encodes individual heart pulse samples according to respiratory cycle stage. A first estimated respiratory waveform of the user during the time window may be generated from the pulse data mapped to the feature space and a plurality of respiratory onsets for the user during the time window calculated based on a plurality of local extrema of the first estimated respiratory waveform. Motion data may also be used, either alone or in a multi-model system, to evaluate user respiration.

According to an aspect of the present disclosure there is provided a computer program product comprising executable code embodied in a non-transitory computer readable medium that, when executing on one or more computing devices, performs the steps of: receiving, from a wearable physiological monitor worn by a user, pulse data including a plurality of heart pulse samples of the user during a time window; mapping the pulse data into a feature space, wherein the feature space encodes individual heart pulse samples according to respiratory cycle stage; generating a first estimated respiratory waveform of the user during the time window from the pulse data mapped to the feature space; and calculating a plurality of respiratory onsets for the user during the time window based on a plurality of local extrema of the first estimated respiratory waveform.

The executable code, when executing on one or more computing devices, may further perform the step of calculating a respiratory rate variability score based on a metric calculated using the plurality of respiratory onsets. The executable code, when executing on one or more computing devices, may further perform the step of determining a physiological condition associated with the user based on the respiratory rate variability score. The physiological condition may be a recovery state of the user. The physiological condition may be a sleep stage of the user. The metric may include one of: a standard deviation of respiratory cycle lengths (RCL) metric; a standard deviation of successive differences of RCL metric; a root mean square of successive differences of RCL metric; a coefficient of variation of RCL metric; a median absolute deviation from median RCL metric; or a coefficient of variation based on an absolute deviation from median RCL metric. The first estimated respiratory waveform may be generated based on a first feature of the feature space, the first feature being related to modulation of heart pulse samples with respect to a respiratory cycle of the user. The step of mapping the pulse data into the feature space may include the step of extracting the first feature from the plurality of heart pulse samples of the pulse data. The first feature may be any one of: a heart pulse frequency variation feature; a heart pulse amplitude variation feature; a steady state baseline wander; or a heart pulse gradient variation feature. The executable code, when executing on one or more computing devices, may further perform the step of generating a second estimated respiratory waveform of the user during the time window from the pulse data mapped to the feature space, where the second estimated respiratory waveform is generated based on a second feature of the feature space, the second feature being related to modulation of heart pulse samples with respect to the respiratory cycle of the user. The step of mapping the pulse data into the feature space may include the step of extracting the second feature from the plurality of heart pulse samples of the pulse data. The plurality of local extrema used to calculate the plurality of respiratory onsets may include local extrema of the first estimated respiratory waveform and local extrema of the second estimated respiratory waveform. The executable code, when executing on one or more computing devices, may further perform the step of determining a weighting for the local extrema, where the plurality of respiratory onsets are calculated according to the weighting. The weighting may be determined by a recurrent neural network. The weighting may be determined based on phase of the first estimated respiratory waveform and the second estimated respiratory waveform. The first estimated respiratory waveform may be generated by interpolating values of the pulse data mapped to the feature space. The values of the pulse data mapped to the feature space may be interpolated using a cubic spline. The values of the pulse data mapped to the feature space may be interpolated at a sampling rate of from between 20 Hz to 1000 Hz. The executable code, when executing on one or more computing devices, may further perform the step of calculating the plurality of local extrema of the first estimated respiratory waveform. The plurality of local extrema may include local maxima of the first estimated respiratory waveform. The pulse data prior may be pre-processed using one or more preprocessing steps prior to mapping the pulse data to the feature space. The one or more preprocessing steps may include at least one of high pass filtering, low pass filtering, pulse quality assessment, and normalizing. The user may be in a steady state during the time window. The plurality of respiratory onsets may be further based on local extrema determined from accelerometer data obtained from the wearable physiological monitor during the time window. Implementations of the described techniques may include hardware, a method or process, or computer software on a computer-accessible medium.

According to a further aspect of the present disclosure, there is provided a method comprising: receiving, from a wearable physiological monitor worn by a user, pulse data including a plurality of heart pulse samples of the user during a time window; mapping the pulse data into a feature space, wherein the feature space encodes individual heart pulse samples according to respiratory cycle stage; generating a first estimated respiratory waveform of the user during the time window from the pulse data mapped to the feature space; and calculating a plurality of respiratory onsets for the user during the time window based on a plurality of local extrema of the first estimated respiratory waveform.

The method may include calculating a respiratory rate variability score based on a metric calculated using the plurality of respiratory onsets. The method may include determining a physiological condition associated with the user based on the respiratory rate variability score. The step of mapping the pulse data may include extracting a first feature from the plurality of heart pulse samples of the pulse data, the first feature being related to modulation of heart pulse samples with respect to a respiratory cycle of the user. The first estimated respiratory waveform may be generated based on the first feature of the feature space. The second estimated respiratory waveform may be generated based on a second feature of the feature space, the second feature being related to modulation of heart pulse samples with respect to the respiratory cycle of the user. The method may include extracting the second feature from the plurality of heart pulse samples of the pulse data. The plurality of local extrema used to calculate the plurality of respiratory onsets may include local extrema of the first estimated respiratory waveform and local extrema of the second estimated respiratory waveform. The plurality of respiratory onsets may be calculated according to the first weighting and the second weighting. Implementations of the described techniques may include hardware, a method or process, or computer software on a computer-accessible medium.

According to an additional aspect of the present disclosure, there is provided a system comprising: a wearable physiological monitor configured to acquire pulse data from a user during a time window, the pulse data including a plurality of heart pulse samples of the user during the time window; and a processor configured to map the pulse data into a feature space, wherein the feature space encodes individual heart pulse samples according to respiratory cycle stage, to generate a first estimated respiratory waveform of the user during the time window from the pulse data mapped to the feature space, and to calculate a plurality of respiratory onsets for the user during the time window based on a plurality of local extrema of the first estimated respiratory waveform.

The processor may be in the wearable physiological monitor. The processor may reside on a remote resource configured to receive data through a data network from the wearable physiological monitor. The wearable physiological monitor may be configured to acquire accelerometer data during the time window, the plurality of respiratory onsets further based on local extrema of the accelerometer data. The time window may correspond to a portion of a sleep session of the user. Implementations of the described techniques may include hardware, a method or process, or computer software on a computer-accessible medium.

### DESCRIPTION OF THE DRAWINGS

The foregoing and other objects, features, and advantages of the devices, systems, and methods described herein will be apparent from the following description of particular embodiments thereof, as illustrated in the accompanying drawings. The drawings are not necessarily to scale, emphasis instead being placed upon illustrating the principles of the devices, systems, and methods described herein. In the drawings, like reference numerals generally identify corresponding elements.

FIG. 1A is a flow chart illustrating a method for respiratory onset detection.

FIG. 1B is a flow chart illustrating further steps that may be performed as part of the method of FIG. 1A.

FIG. 2 illustrates features that may be extracted from a heart pulse sample.

FIG. 3 illustrates steady state baseline wander features that may be extracted from pulse data.

FIG. 4A provides an illustrative example of respiratory onset detection from heart pulse data.

FIG. 4B provides an illustrative example of respiratory onset detection from heart pulse data.

FIG. 4C provides an illustrative example of respiratory onset detection from heart pulse data.

FIG. 4D provides an illustrative example of respiratory onset detection from heart pulse data.

FIG. 4E provides an illustrative example of respiratory onset detection from heart pulse data.

FIG. 5A shows a machine learning model for weighting respiratory onsets.

FIG. 5B shows an encoder-decoder network.

FIG. 6 shows a portion of a system for respiratory rate onset estimation and physiological condition detection.

FIG. 7 is a flow chart illustrating a method for calculating respiratory onsets of a user.

FIG. 8A illustrates accelerometer based respiratory onset detection.

FIG. 8B illustrates accelerometer based respiratory onset detection.

FIG. 8C illustrates accelerometer based respiratory onset detection.

FIG. 9 shows a physiological monitoring device.

FIG. 10 illustrates a physiological monitoring system.

FIG. 11 shows a smart garment system.

FIG. 12 is a block diagram of a computing device.

### DETAILED DESCRIPTION

The embodiments will now be described more fully hereinafter with reference to the accompanying figures, in which preferred embodiments are shown. The foregoing may, however, be embodied in many different forms and should not be construed as limited to the illustrated embodiments set forth herein. Rather, these illustrated embodiments are provided so that this disclosure will convey the scope to those skilled in the art.

Information or data pertaining to the respiratory cycles (or breathing cycles) of a user may be useful for diagnosis and monitoring of respiratory conditions, early detection of respiratory distress, and/or personalized care (e.g., helping aid user recovery, improve sleep, providing feedback on breathing technique during low motion activities such as meditation, etc.). However, obtaining such information or data outside of a clinical setting is difficult and may not be readily possible without invasive techniques or specialized equipment. The present disclosure provides non-invasive techniques for recovering information regarding a user's respiratory cycles from heart pulse data that may be easily and non-invasively obtained from a user's personal device, such as a wearable physiological device worn by the user. This opens up a rich vein of data to be generated from which insights into the user's physiological condition may be efficiently and effectively obtained. The user is thus provided with “at home” insights into their respiratory state and/or physiological condition without requiring specialized medical equipment or the performance of invasive techniques.

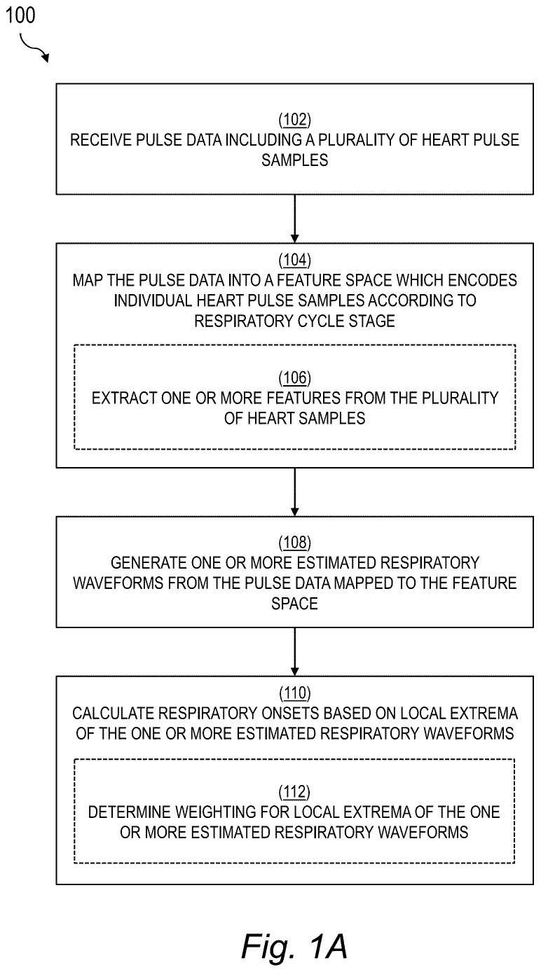

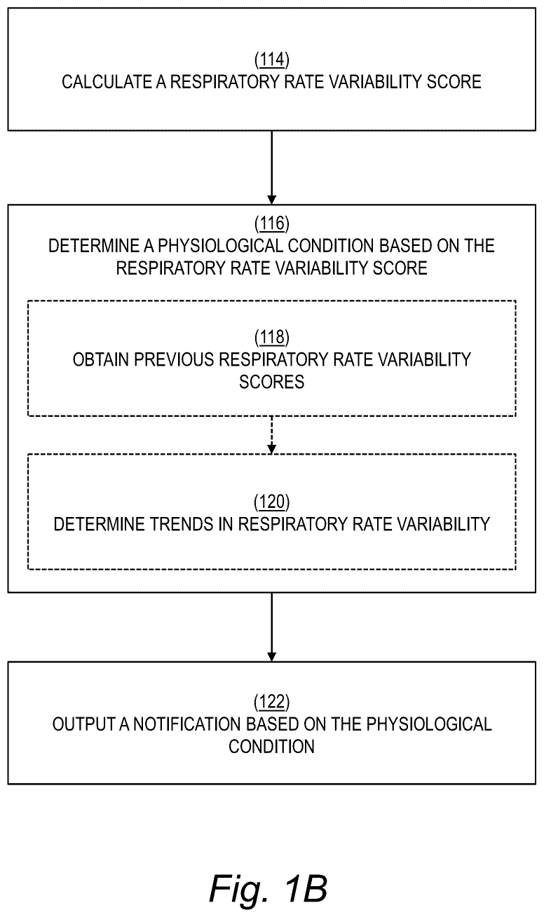

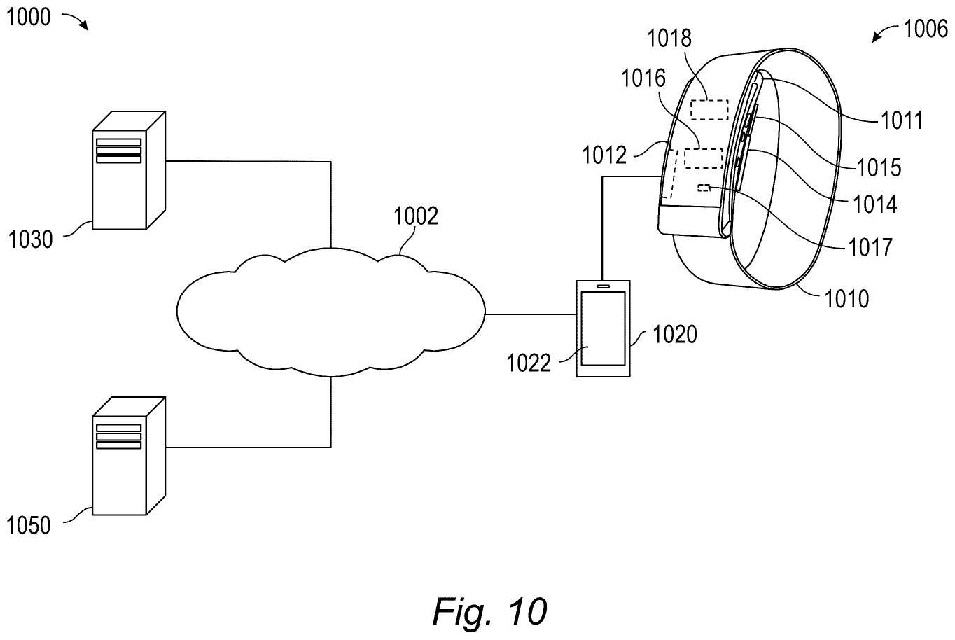

FIG. 1A is a flow chart illustrating a method 100 for respiratory onset detection for a user of a wearable physiological monitor. The method 100 may be used in cooperation with any of the devices, systems, and methods described herein, such as by a user device (e.g., a mobile device) that is communicatively coupled to a wearable, continuous physiological monitoring device. For example, the one or more user devices 1020 that are communicatively coupled to the physiological monitor 1006 in FIG. 10. In general, the method 100 maps a user's heart pulse data into a feature space that encodes individual heart pulse samples according to respiratory cycle stage. One or more estimated respiratory waveforms of the user are then constructed from the pulse data mapped to the feature space. Extrema of the estimated respiratory waveforms can then be used as estimates of respiratory onsets for the user. That is, the user's pulse data may latently encode the user's breathing patterns in a relationship referred to as respiratory sinus arrhythmia—the manner in which pulse rate changes due to cycles of inhalation and exhalation. The pulse data can be mapped into a feature space that recovers this latent encoding. As such, the method 100 provides an efficient, accurate, and non-invasive approach for estimating respiratory onset of a user from heart pulse data. The respiratory onset information estimated by the method 100 may be subsequently used to calculate physiological metrics that indicate the respiratory state and/or physiological condition of the user.

As shown in step 102, the method 100 may include receiving, from a wearable physiological monitor worn by a user, pulse data including a plurality of heart pulse samples of the user during a time window. For example, the pulse data may be obtained from a physiological monitor such as any of the physiological monitors described herein. More particularly, the sequence of pulse data may be obtained from a suitable sensor (e.g., a photoplethysmography (PPG) sensor or an electrocardiogramsor) coupled to the physiological monitor.

The pulse data comprises a sequence of pulses (heart beats or heart pulse samples) that relate to the cardiac activity of the user during the time window. That is, the pulse data corresponds to a waveform or time series of values characterizing the cardiac activity of the user during the time window. The time window may be set to a predetermined length of time (e.g., 30 s, 60 s, 120 s, etc.) or may be dynamically determined such that the time window is of sufficient length for the plurality of heart pulse samples captured within the time window to satisfy a threshold quality criterion. The user may be in a steady state during the time window. When in a steady state, the user's physiological processes and body are relatively stable and consistent over a period of time (e.g., the time window), with vital signs like heart rate, breathing rate, and body temperature maintaining consistent levels. This state of equilibrium means the user's body is balanced, with no significant stressors or fluctuations in metabolic processes. Additionally, in a steady state the user is not actively responding to external stimuli, allowing the body to maintain a steady internal physiology.

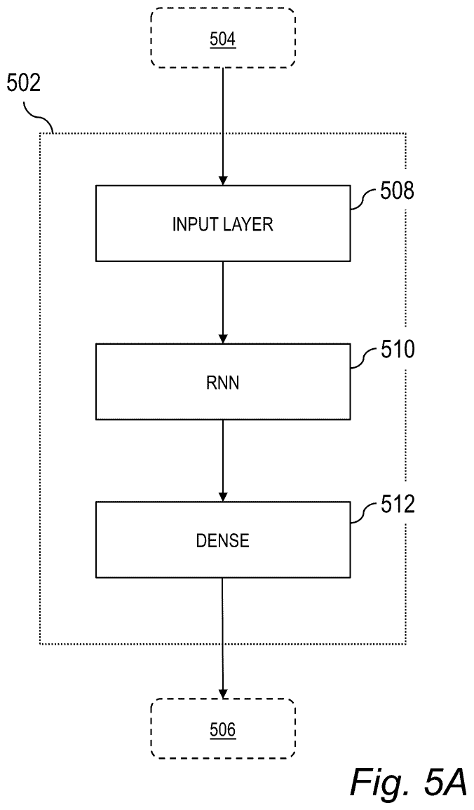

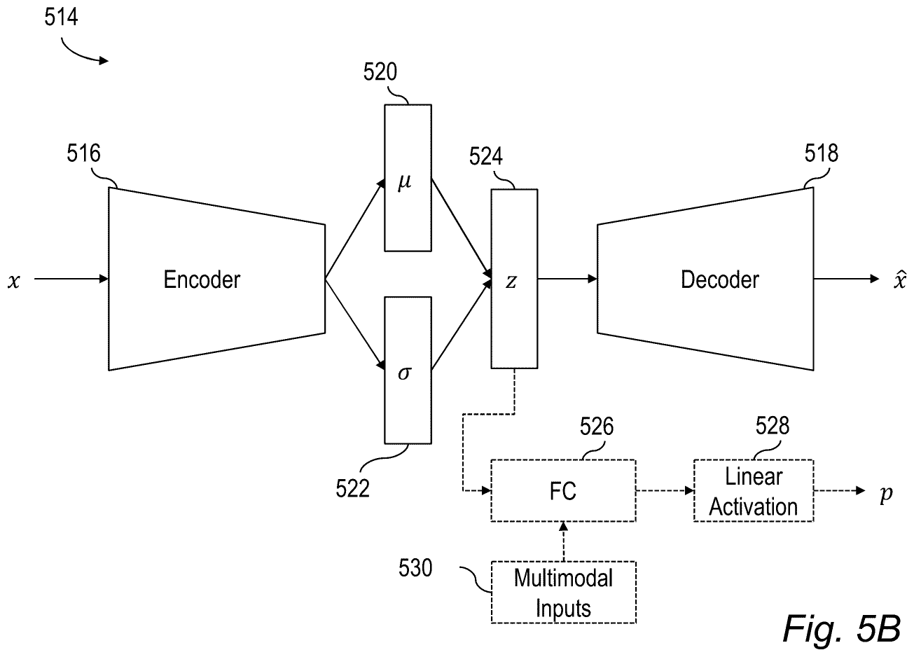

In one example implementation, the pulse data may be obtained while the user is asleep, although the pulse data may be received by a processing resource during sleep, or stored on the wearable device and transmitted to a remote processing resource after sleep has ended. In one embodiment, pulse data consistency may be improved by obtaining training data at a particular point in sleep. Thus, for example, stages of sleep may be tracked using other data, such as a heart rate, skin temperature, and motion, and used to identify a particular stage of sleep so that a particular stage may be used for sampling pulse data. For example, slow wave or deep sleep presents an opportunity for consistent measurements of pulse data and may be used as a criterion for pulse selection. Still greater consistency may be achieved by sampling during a particular episode of deep sleep (e.g., the last deep sleep episode during a sleep interval) or during a particular time within deep sleep (e.g., the middle of an episode, near the end of an episode, or at a highest data quality portion of an episode). In one aspect, training data may be exclusively selected based on one or more sleep criteria, and the same selection criteria may be used to acquire data for subsequent inferencing with a machine learning model or the like. This approach is not necessary but can help to mitigate extraneous influences on pulse shape and help to ensure that the latent space features encoded by a machine learning model are matched to inferencing data acquired under similar or identical physiological conditions for a user. In another aspect, this can advantageously facilitate concurrency, or at least consistency, with motion data used for motion-based inferencing as described below, e.g., so that inferences based on motion can be chronologically and/or physiologically associated with inferences based on pulse data when performing multi-model respiration evaluations. In one aspect, a pulse quality check may be performed to determine whether the user is in a steady internal state such as sleep, e.g., using an autoencoder based approach such as that described in relation to FIG. 5B below.

The pulse data may be pre-processed using one or more preprocessing steps (e.g., prior to being received at step 102 or after the pulse data is received at step 102 but before step 104). The one or more preprocessing steps may include high pass filtering of the pulse data. High pass filtering allows high-frequency components of the pulse data to pass through the filter while attenuating or reducing the low-frequency components and any DC offset. High pass filtering helps eliminate unwanted low-frequency noise while helping to enhance the detection of sudden changes or events within the pulse data. The one or more preprocessing steps may also or instead include low pass filtering of the pulse data. Low pass filtering allows low-frequency components of the pulse data to pass through while attenuating or reducing the high-frequency components, e.g., to remove spurious measurements or noise that is not likely associated with physiological processes. Low pass filtering helps remove unwanted high-frequency noise while helping to enhance the detection of slow-varying components in the pulse data. In one aspect, bandpass filtering may be used to suppress signals outside one or more frequency bands of interest. The one or more preprocessing step may include normalizing the pulse data, e.g., to adjust the amplitude of the pulse data so that it falls within a specific range (e.g., between 0 and 1 or between −1 and +1). The one or more preprocessing steps may include a detrending step. Detrending may remove trends that represent long-term changes in the pulse data thereby helping to highlight or suppress cyclical, seasonal, or irregular components of the pulse data. The detrending step may be performed by techniques such as differencing, regression modelling, and/or moving average filters. The one or more preprocessing steps may include a pulse quality assessment step where pulses are filtered and/or selected such that pulses that satisfy one or more criteria are maintained for further analysis. Alternatively, if the pulse data comprises at least one pulse that does not satisfy the one or more criteria then the pulse data may not be used for analysis (and a further sequence or segment of pulse data spanning a different or subsequent time window is obtained). The criteria may include a quality criterion based on an expected or model pulse shape. For example, the quality criterion may be satisfied when a pulse is determined to have one or more pulse characteristics such as a dicrotic notch. A dicrotic notch classifier (e.g., as described in relation to FIG. 5B below) or other tool may be used to assign a dicrotic notch extent score to each pulse within the pulse data and pulses having a dicrotic notch extent score above a threshold value (e.g., a score that indicates that the pulse has a dicrotic notch with a probability of 0.5, 0.75, 0.9, 0.95, or the like) may be maintained for further analysis.

As shown in step 104, the method 100 may include mapping the pulse data into a feature space which encodes individual heart pulse samples according to respiratory cycle stage.

In general, the pulse data is transformed at step 104 by mapping heart pulse samples (e.g., individual heart beats) into a feature space that characterizes individual heart pulse samples in relation to respiratory cycle stage. For example, the feature space characterizes or encodes a heart pulse sample occurring at the start of a respiratory cycle (e.g., during inhalation) differently than a heart pulse sample occurring at the end of the respiratory cycle (e.g., during exhalation). Here, a heart pulse sample being characterized or encoded within the feature space means that a quantitative value is assigned to the heart pulse sample based on the position of the heart pulse sample mapped to the feature space. As such, the feature space recovers hidden, or latent, information regarding the user's respiratory patterns from their heart pulse data thereby enabling respiratory data to be obtained for the user without needing additional equipment, sensors, or invasive techniques.

The heart pulse sample may be mapped to the feature space using a feature transformation approach such as by use of a machine learning model. For example, a dimensionality reduction or manifold learning approach may be used to transform each heart pulse sample into a low-dimensional feature space. Morphological features may be extracted from each heart pulse sample and mapped into the low-dimensional feature space (e.g., 1-dimensional) by an algorithm such as principal components analysis (PCA), Isomap, UMAP, or the like. In another example, an autoencoder (encoder-decoder) may be trained to map the heart pulse data into a low-dimensional latent feature space that characterizes heart pulse samples according to respiratory cycle stage.

A heart pulse sample may be mapped to the feature space using a feature extraction approach. That is, one or more features are extracted from a heart pulse sample and the heart pulse sample is represented within the feature space by the combination of the one or more extracted features. The feature(s) extracted from a heart pulse sample characterize the modulation, or change, of the heart pulse sample in relation to a respiratory cycle. As such, to map a heart pulse sample into the feature space, the feature extraction approach extracts one or more modulation features from the heart pulse sample. The one or more modulation features include frequency modulation, amplitude modulation, and gradient modulation. Frequency modulation captures, or estimates, the frequency variation of the pulse data that occurs due to respiratory-induced frequency variation (e.g., the variation in time between two consecutive heartbeats during inspiration and expiration). Amplitude modulation captures, or estimates, the amplitude variation of the pulse data that occurs due to respiratory-induced amplitude variation (e.g., the variation in pulse amplitude during inspiration and expiration). Gradient modulation captures, or estimates, the variation in the pulse gradient that increases during inspiration and decreases during expiration. Advantageously, the feature extraction approach provides a computational resource efficient mechanism for mapping the pulse data to a feature space that may be particularly advantageous on low-resource or low-power devices.

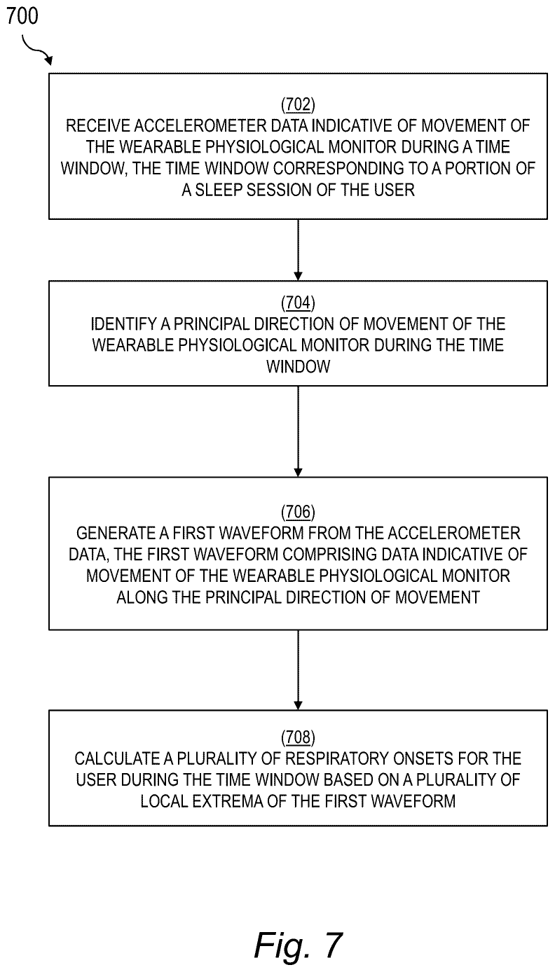

Step 104 may include step 106, where one or more features are extracted from the plurality of heart pulse samples of the pulse data, the one or more features being related to modulation of heart pulse samples with respect to a respiratory cycle of the user. As stated above, a feature extracted from a heart pulse sample characterizes the heart pulse sample in relation to where in a respiratory cycle of the user the heart pulse sample occurs. That is, heart pulses of a user exhibit different morphological and temporal characteristics at different time points of a respiratory cycle of the user; the features extracted from a heart pulse quantify, or characterize, these morphological characteristics thereby enabling respiratory data to be approximated from heart pulse data without requiring additional equipment or reversion to invasive techniques. That is, the user may monitor their changes in respiratory patterns through heart pulse data obtained using a wearable physiological monitor such as those shown in FIGS. 7-9.

Step 106 may include extracting a first feature from the plurality of heart pulse samples of the pulse data. That is, for one or more pulses within the plurality of heart pulse samples, one or more first features are extracted. Also, or instead, a feature may be extracted for each/all heart pulse sample(s) within the plurality of heart pulse samples. The plurality of heart pulse samples may be segmented into individual heart pulse samples by utilizing a peak finding algorithm on the inverted segment of pulse data (e.g., the inverted PPG signal). The peaks identified using the peak finding algorithm are then used to define the boundaries between each pulse within the segment of pulse data. An example peak finding algorithm is the local maxima algorithm based on a comparison of neighboring values.

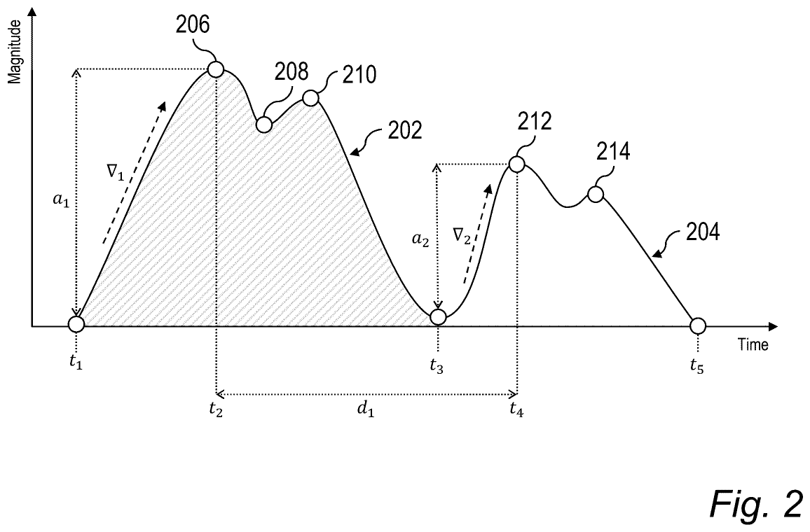

The first feature may be a heart pulse frequency variation feature (alternatively referred to as a heart pulse frequency feature, an inter-pulse duration feature, an RR interval feature, or a peak-to-peak distance feature). A heart pulse frequency variation value may be extracted from the pulse data by calculating the peak-to-peak distance between successive or consecutive heart pulse samples in the plurality of heart pulse samples. For example, for a first heart pulse sample that achieves maximum amplitude at time point tₐ, the heart pulse frequency variation feature for the first heart pulse sample is |tₐ-t_{b}| where t_{b} is the time point at which the next heart pulse sample (i.e., the heart pulse sample that occurs immediately after the first heart pulse sample) achieves maximum amplitude. This is described in more detail in relation to FIG. 2 below. The heart pulse frequency variation feature captures the respiratory-induced frequency variation (RIFV) of the pulse data that occurs due to the interval between successive heartbeats (often referred to as the “RR interval”) decreasing during inspiration (inhalation) and increasing during expiration (exhalation).

The first feature may be a heart pulse amplitude variation feature (alternatively referred to as a heart pulse amplitude feature, an amplitude feature, a systolic amplitude feature, or a systolic variation feature). A heart pulse amplitude variation feature may be extracted from a heart pulse sample by calculating the absolute difference between the amplitude of the heart pulse sample at the start of the pulse and the maximum amplitude of the heart pulse sample (as described in more detail in relation to FIG. 2 below). This feature captures the respiratory-induced amplitude variation (RIAV) of the pulse data that occurs due to the amplitude of heart pulse samples decreasing during inspiration (inhalation) and increasing during expiration (exhalation).

The first feature may be a heart pulse gradient variation feature (alternatively referred to as a heart pulse gradient feature, a heart pulse rate of change feature, or a gradient/rate of change feature). A heart pulse gradient variation feature value may be extracted from a heart pulse sample by calculating the maximum value of the first derivative of the heart pulse sample. This feature captures the variation arising from the pulse gradient increasing during inspiration (inhalation) and decreasing during expiration (exhalation).

The first feature may be a steady state baseline wander feature (alternatively referred to as a baseline wander or detrending feature). A steady state baseline wander feature may be extracted from a heart pulse sample by calculating the difference between a representative magnitude value of the heart pulse sample and a baseline magnitude value. The baseline magnitude value may be an average magnitude value calculated from all heart pulse samples in the pulse data or a fixed value such as 0. The representative magnitude value of the heart pulse sample may be the average magnitude value of the heart pulse sample or the magnitude value of a predetermined point in the heart pulse (e.g., the systolic peak, the dicrotic notch, the diastolic peak, etc.). The steady state baseline wander feature encodes the degree to which the heart pulse sample has deviated, or wandered, from a baseline value. Typically, the magnitude of a heart pulse sample is greater than a baseline (e.g., average) value during inspiration (inhalation) and less than the baseline value during expiration (exhalation).

As a heart pulse sample may be characterized by more than one of the above-described features, step 106 may include extracting a second feature from the plurality of heart pulse samples of the pulse data. The second feature may be a heart pulse frequency variation feature, a heart pulse amplitude variation feature, a steady state baseline wander of heart pulse feature, or a heart pulse gradient variation feature (as described above). That is, multiple features may be extracted for each heart pulse sample in the pulse data. For example, step 106 may include extracted a heart pulse frequency variation feature and a heart pulse amplitude variation feature for each heart pulse sample in the plurality of heart pulse samples. Step 106 may further include extracting a third feature and/or extracting a fourth feature from the plurality of heart pulse samples of the pulse data.

As shown in step 108, the method 100 may include generating one or more estimated respiratory waveforms of the user during the time window from the pulse data mapped to the feature space.

In general, an estimated respiratory waveform corresponds to an approximate reconstruction of the user's respiratory waveform (i.e., a representation of the user's breathing cycle representing changes in pressure/volume/air flow in and out of the user's lungs over time) from pulse data of the user. The one or more estimated respiratory waveforms may be generated by interpolating values of the pulse data mapped to the feature space. The mapped values may be interpolated to reconstruct a waveform representing the estimated respiratory waveform—i.e., breathing cycle—of the user over the time period. An estimated respiratory waveform may be interpolated using any suitable interpolation technique (e.g., linear interpolation, polynomial interpolation, cubic interpolation, Hermite interpolation, etc.). In one implementation, a cubic spline interpolation approach is used to generate an estimated respiratory waveform. The values of the pulse data mapped to the feature space are interpolated at a sample rate of from between 20 Hz to 1000 Hz, from between 100 Hz to 500 Hz, from between 200 Hz to 300 Hz, or at 256 Hz. An estimated respiratory waveform may be generated by interpolating the pulse data mapped to the feature space across all dimensions (or features) of the feature space. For example, if 2 features are extracted from the pulse data, then an estimated respiratory waveform (or multidimensional waveform) may be generated by performing trilinear or tricubic interpolation between each point (which is represented by a time point and two feature values). Alternatively, and as described below, estimated respiratory may be independently generated for each feature.

The one or more estimated respiratory waveforms may include a first estimated respiratory waveform. As such, the method 100 may include the step of generating the first estimated respiratory waveform of the user during the time window from the pulse data mapped to the feature space. The first estimated respiratory waveform may be generated based on a first feature of the feature space that is related to modulation of heart pulse samples with respect to a respiratory cycle of the user (as described above). The first estimated respiratory waveform may then be generated by interpolating values of the pulse data mapped to a first feature of the feature space. For example, if heart pulse amplitude variation features are extracted from 30 heart pulse samples (at 30 corresponding time points), then the 30 mapped values within the feature space are interpolated to reconstruct or estimate the respiratory waveform (i.e., interpolate the missing values between the 30 corresponding time points within the feature space).

The one or more estimated respiratory waveforms may include a second estimated respiratory waveform. As such, the method 100 may include the step of generating the second estimated respiratory waveform of the user during the time window from the pulse data mapped to the feature space. The second estimated respiratory waveform may be generated based on a second feature of the feature space that is related to modulation of heart pulse samples with respect to a respiratory cycle of the user. The second feature of the feature space is different to the first feature of the feature space. For example, the first feature may be heart pulse frequency variation, and the second feature may be heart pulse amplitude variation. The second estimated respiratory waveform may then be generated by interpolating values of the pulse data mapped to a second feature of the feature space. In a similar way, the one or more estimated respiratory waveforms may include a third or a fourth estimated respiratory waveform generated in the same manner from a third or fourth feature of the feature space respectively. In one implementation, the one or more estimated respiratory waveforms include a first estimated respiratory waveform generated from the pulse data mapped to a heart pulse frequency variation feature, a second estimated respiratory waveform generated from the pulse data mapped to a heart pulse amplitude variation feature, and a third estimated respiratory waveform generated from the pulse data mapped to a heart pulse gradient variation feature. In a further implementation, the one or more estimated respiratory waveforms further include a fourth estimated respiratory waveform generated from the pulse data mapped to a steady state baseline wander of heart pulse feature.

As shown in step 110, the method 100 may include calculating a plurality of respiratory onsets for the user during the time window based on a plurality of local extrema of the one or more estimated respiratory waveforms. The estimated respiratory waveforms characterize the respiratory cycles of the user during the time window and so the local extrema of these waveforms may be used to identify the onset, or start, of a respiratory cycle.

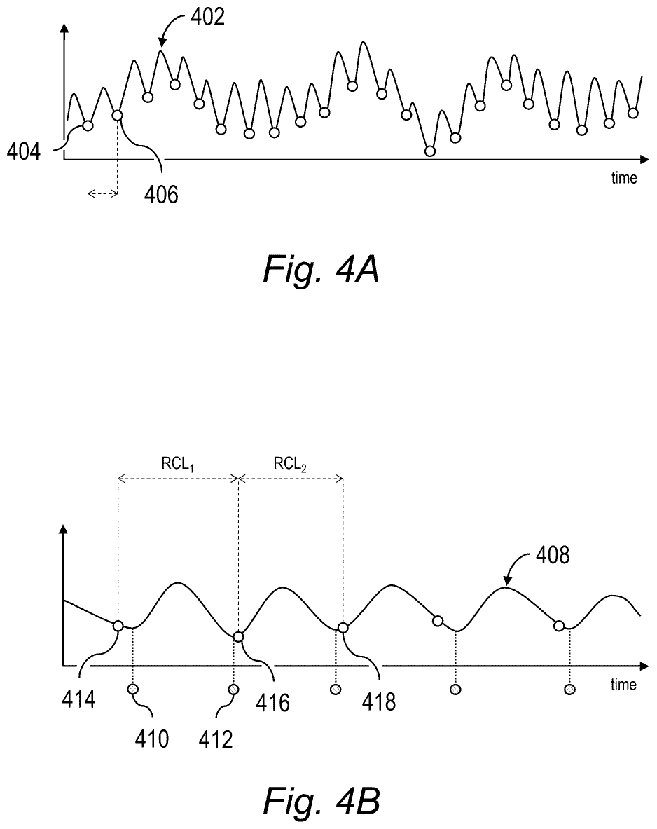

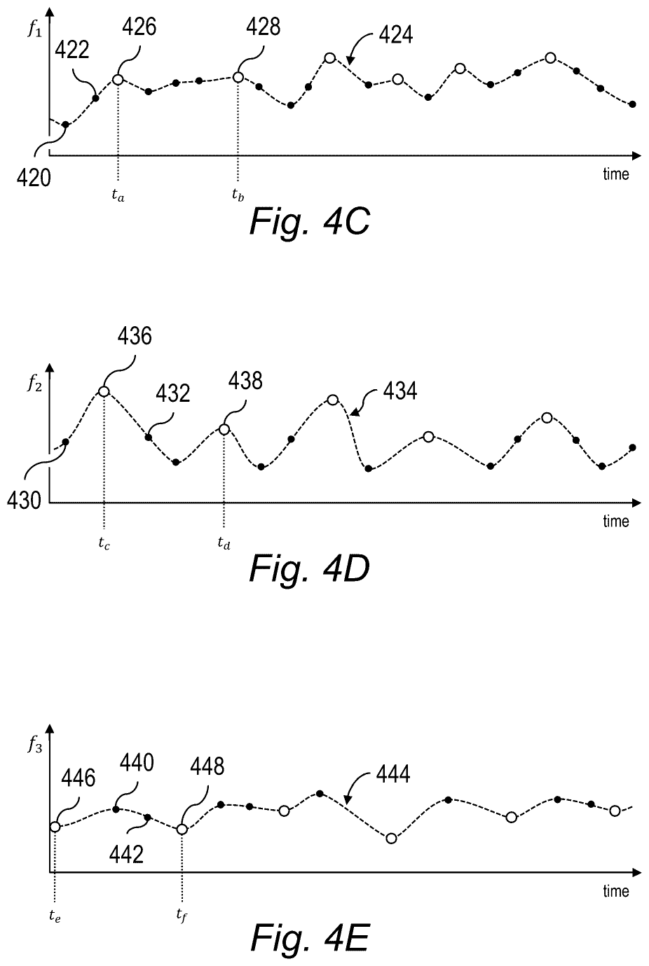

Step 110 may include calculating the plurality of local extrema of the one or more estimated respiratory waveforms (e.g., the local extrema of the first estimated respiratory waveform and/or the local extrema of the second estimated respiratory waveform, etc.). The local extrema may be local maxima or local minima of the one or more estimated respiratory waveforms. The determination of whether maxima or minima are calculated depends on the features, or feature space, from which the estimated respiratory waveform is generated. Since heart pulse frequency variation and heart amplitude frequency variation decreases during inhalation and increases during exhalation, the estimated respiratory waveforms generated from these features may be considered to be out of phase with the respiratory cycle of the user. The local maxima of these waveforms are therefore used to identify respiratory onsets. Conversely, since heart pulse gradient variation increases during exhalation and increases during inhalation, the estimated respiratory waveform generated from this feature is in phase with the user's respiratory cycle and so the local minima of this waveform are used to identify respiratory onsets. This is illustrated in FIGS. 4A-4E as described in more detail below.

Step 110 may include step 112 where weightings for local extrema of the one or more estimated respiratory waveforms are determined such that the plurality of respiratory onsets are calculated according to the weightings. Weightings may be used when multiple respiratory onsets are determined for a single heart pulse or when multiple respiratory onsets appear within a predetermined window (e.g., 0.5 s, 1 s, 2 s, 4 s, etc.). The multiple respiratory onsets may then be combined or aggregated according to the weightings such that the estimated respiratory onset is a weighted combination of the multiple respiratory onsets.

For example, step 112 may comprise determining a first weighting for the local extrema of the first estimated respiratory waveform and determining a second weighting for the local extrema of the second estimated respiratory waveform. The plurality of respiratory onsets may then be calculated according to the first weighting and the second weighting. The weightings may be determined by a machine learning model such as a neural network or a recurrent neural network (RNN). An example RNN for determining weightings is described in more detail in relation to FIG. 5A below. Alternatively, the weightings may be determined based on phase information. That is, the weighting may be determined based on a phase of the first estimated respiratory waveform and a phase of the second estimated waveform.

The plurality of local extrema may further include local extrema determined from accelerometer data of the wearable physiological monitor (e.g., as determined using the method 700 shown in FIG. 7 and described below). As such, the local extrema calculated from both heart rate data and accelerometer data may be combined (e.g., weighted as described above) to determine an accurate and robust indicator of respiratory onsets for the user. Advantageously, multiple different sensor data may be combined or fused to provide a more robust and accurate estimate of the user's respiratory onsets.

FIG. 1B is a flow chart illustrating further steps that may be performed as part of the method 100 of FIG. 1A. The steps shown in FIG. 1B utilize the respiratory onsets calculated using the steps described in FIG. 1A to determine a physiological condition associated with the user. The respiratory onsets may also or instead be respiratory onsets determined using the method 700 of FIG. 7. More particularly, the respiratory onsets are used to calculate a respiratory rate variability score for the user that can be used to identify physiological conditions such as recovery rate and/or sleep stage. As such, there is provided an efficient, accurate, and non-invasive approach for estimating respiratory related physiological conditions associated with the user from heart pulse data.

As shown in step 114, the method 100 may include calculating a respiratory rate variability score based on a metric calculated using the plurality of respiratory onsets. As such, the step 114 may be performed after the step 110 shown in FIG. 1A and/or after the step 708 shown in FIG. 7. Respiratory rate variability refers to the natural fluctuations in a user's rate of breathing over time. The respiratory rate variability (RRV) score quantifies the estimated respiratory rate variability of the user during the time window from the plurality of respiratory onsets. The RRV score may be calculated using one or more metrics and the plurality of respiratory onsets. In general, a metric (or RRV metric) returns a quantitative value, or score, that is indicative of, or associated with, the respiratory rate variability of the user during the time window. A metric returns a quantitative value, or score, based on the plurality of respiratory onsets calculated from the pulse data of the user during the time window.

The metric may be a standard deviation of respiratory cycle length metric. Respiratory cycle length (RCL) is the length (in time, e.g., seconds, milliseconds, etc.) of a single respiratory cycle. RCL may be calculated as the absolute difference between consecutive respiratory onsets and may be alternatively referred to as breath-to-breath interval (BBI). For example, if a first respiratory onset is identified at time point tₐ and the next respiratory onset is identified at time point t_{b}, then these two respiratory onsets bound a single respiratory cycle having an RCL of |tₐ-t_{b}|. The plurality of respiratory onsets determined at step 110 of the method 100 may therefore be used to generate one or more RCL values (referred to herein as the set of RCL values). The standard deviation of RCL length metric may be calculated as the standard deviation of the set of RCL values.

The metric may be a standard deviation of successive differences (SDSD) of RCL metric. Given two successive respiratory cycles with RCL values of RCL₁=|tₐ-t_{b}| and RCL₂=|t_{b}-t_{c}|, the successive difference between the two respiratory cycles is |RCL₁-RCL₂|. Given the set of RCL values (generated as described above), a set of successive differences of RCL values may then be calculated and the standard deviation of this set calculated as the SDSD of RCL metric. Advantageously, the SDSD metric helps capture local changes in the user's breathing pattern over the time window.

The metric may be a root mean square of successive differences (RMSSD) of RCL metric. The metric may be calculated by taking the square root of the mean value of the set of squared successive differences (calculated as described above).

The metric may be a coefficient of variation of RCL metric. The coefficient of variation is calculated by normalizing the standard deviation of the set of RCL values relative to the mean value of the set of RCL values. Advantageously, the coefficient of variation is a scale free measure that allows for comparison across individuals or populations with differing average RCL values.

The metric may be a median absolute deviation from median (MADM) RCL metric. This metric measures the variability of RCL values around the median RCL value. The MADM RCL metric may be calculated as the median of the absolute differences between the RCL values in the set of RCL values and the median value of the set of RCL values. Advantageously, the MADM RCL metric provides a robust measure of variability that is less sensitive to noise and outlier values.

The metric may be a coefficient of variation based on an absolute deviation from median RCL metric. This metric may be calculated by normalizing the MADM RCL metric relative to the median value of the set of RCL values.

As shown in step 116, the method 100 may include determining a physiological condition associated with the user based on the respiratory rate variability (RRV) score. The RRV score provides a quantitative and comparable representation of the respiratory state of the user during the time window and so may be used to determine a physiological condition associated with the user, such as a recovery state or a sleep stage.

In one example, a recovery state of the user may be determined from the RRV score. For example, based on the user's RRV score determined during a night's sleep, a recovery state may be determined and represented as a percentage (e.g., a recovery score where a lower percentage indicates that the user is still in a recovery state whereas a higher percentage indicates that the user is recovered and ready to take on exercise/strain). The recovery state may be directly based on the user's RRV; for example, a low RRV score may indicate a high level of recovery and a high RRV score may indicate a low level of recovery. Also, or instead, the recovery state may be determined as a weighted combination of the user's RRV score along with other physiological metrics such as heart rate variability, sleep score, and recent strain.

In one example, a sleep stage of the user may be determined from the RRV score using a sleep stage prediction model. The sleep stage prediction model may be rule based where the RRV score is compared against threshold values to determine sleep stage. For example, if the RRV is below a first threshold, then it is predicted that the user was/is in a stage 3 (deep sleep) non-REM sleep stage during the time window, and if the RRV is above the first threshold but below a second threshold then it is predicted that the user was/is in a stage 2 non-REM sleep stage during the time window. The sleep stage prediction model may be a machine learning model trained to map from RRV score to sleep stage. For example, a training data set of RRV scores and associated sleep stage labels (e.g., 0 for REM sleep, 1 for stage 1 non-REM sleep, 2 for stage 2 non-REM sleep, etc.) may be used to train a classifier to predict a sleep stage label from a given RRV score. The skilled person will appreciate that any suitable classifier may be used such as a decision tree classifier, a Random Forest classifier, a multilayer perceptron, and the like.

Step 116 may include step 118 where a set of previous respiratory rate variability scores of the user are obtained. The set of previous RRV scores may have been previously calculated for the user over a range of time windows. The range of time windows may collectively represent a single event (e.g., a single night's sleep) or time windows over a longer time period (e.g., over 2 days, 4 days, 1 week, 1 month, etc.). The set of previous RRV scores may be obtained from a storage location such as a memory or persistent memory of a device (e.g., the one or more devices 1020 shown in FIG. 10) and may be transmitted to the device or server performing the method 100.

After step 118, step 116 may include step 120 where one or more trends in the respiratory rate variability of the user are determined. The set of previous RRV scores may be compared, analyzed, or otherwise processed to determine one or more trends or changes in the user's RRV. For example, the set of RRV scores obtained for a user over a single night's sleep may be analyzed to identify specific times or periods in which the user reached a certain sleep stage (e.g., when they were in deep sleep and for how long). As a further example, the set of RRV scores obtained for a user over a period of 1 month may be analyzed to identify a change in the RRV, recovery rate, or the like of the user of the 1 month period. Such trends provide the user with non-invasive feedback into their longitudinal physiological condition/state that can aid in their recovery and/or help drive longer term health improvements.

As shown in step 122, the method 100 may include outputting a notification based on the physiological condition. The notification may include information based on the physiological condition, the RRV score, and/or the respiratory onsets. For example, the notification may take the form of a notification, prompt, or information screen displayed on a device of the user (e.g., the one or more devices 1020 shown in FIG. 10) and including the information (e.g., “your recovery rate is XX %”). In one example, the notification includes information based on the one or more trends in the RRV of the user. For example, the notification may include information detailing sleep stage information for the user over one or more nights of sleep. As a further example, the notification may include a graph or chart of recovery rate of the user over a time period (e.g., 2 days, 4 days, 1 week, etc.).

The notification may also take the form of haptic feedback provided to the user via a device (e.g., the one or more devices 1020 or the wearable physiological monitor 1006 shown in FIG. 10). The notification may be provided to the user if one or more conditions based on the RRV score are met. For example, if the user's recovery rate is above a threshold level then haptic feedback may be provided to the user to indicate that their threshold recovery rate has been reached.

FIG. 2 illustrates features that may be extracted from a heart pulse sample. FIG. 2 shows a first heart pulse sample 202 and a second heart pulse sample 204. The first heart pulse sample comprises a systolic peak 206, a dicrotic notch 208, and a diastolic peak 210. Also shown in FIG. 2 are a systolic peak 212 and a diastolic peak 214 of the second heart pulse sample 204.

A pulse or heartbeat, such as the first heart pulse sample 202 or the second heart pulse sample 204, is generally composed of two components-a systolic component followed by a diastolic component. The systolic component arises from a forward-going pressure wave along the left ventricle while the diastolic component arises from pressure wave transmitted along the aorta (Millasseau, S. C., Kelly, R., Ritter, J., and Chowienczyk, P. (2002). Determination of age-related increases in large artery stiffness by digital pulse contour analysis. Clin. Sci. 103, 371-377). The systolic peak of the systolic component (e.g., the systolic peak 206 of the first heart pulse sample 202) corresponds to the point in the pulse that represents the maximum blood volume during each cardiac cycle. Typically, the systolic peak corresponds to the point within a pulse that has the largest amplitude. In the example shown in FIG. 2, the ascension point of the first heart pulse sample 202 begins at time point t₁ where the first heart pulse sample 202 begins to rise from a baseline level. The systolic peak 206 of the first heart pulse sample 202 occurs at time t₂ and has amplitude α₁ (measured in relation to the amplitude of the first heart pulse sample 202 at the ascension point, i.e., at time point t₁). The diastolic peak of the diastolic component (e.g., the diastolic peak 210 of the first heart pulse sample 202) corresponds to the highest amplitude of a pulse during the diastolic component following the systolic peak. The dicrotic notch (e.g., the dicrotic notch 208 of the first heart pulse sample 202) corresponds to the inflection point between a pulse's systolic peak and diastolic peak. The descension point of a pulse corresponds to the point at which the pulse has reached a baseline level (e.g., at the end of a heartbeat). In the example shown in FIG. 2, the descension point of the first heart pulse sample 202 occurs at time point t₃, that also corresponds to the ascension point, or start, of the second heart pulse sample 204.

The peak-to-peak distance of a pulse (or RR interval) may be used to calculate a heart pulse frequency variation feature (as described above) and may be calculated using the systolic peaks of consecutive pulses. In the example shown in FIG. 2, the peak-to-peak distance for the first heart pulse sample 202 may be calculated as the absolute difference between the time of the systolic peak 206 of the first heart pulse sample 202, t₂, and the time of the systolic peak 212 of the second heart pulse sample 204, t₄. That is, the peak-to-peak distance associated with the first heart pulse sample 202 may be calculated as d₁=t₄−t₂. Similarly, while not shown in FIG. 2, the peak-to-peak distance for the second heart pulse sample 204 may be calculated from the absolute difference between the time of the systolic peak 212 of the second heart pulse sample 204 and the time of the systolic peak of the next or subsequent (consecutive) heart pulse sample.

The amplitude of a pulse may be calculated as the difference between the amplitude of the heart pulse sample at the start of the pulse and the maximum amplitude of the heart pulse sample. For the first heart pulse sample 202, the amplitude may be calculated as the absolute difference in amplitude/magnitude of the pulse between time point t₁ (when the heart pulse begins and has lowest amplitude) and time point t₂ (when the first heart pulse sample 202 has greatest amplitude, i.e., the systolic peak 206). Similarly, the amplitude of the second heart pulse sample 204 may be calculated as the absolute difference in amplitude between time point t₃ (i.e., the ascension point of the second heart pulse sample 204) and time point t₄ (when the second heart pulse sample 204 has greatest amplitude, i.e., the systolic peak 212).

The gradient of a pulse may be calculated by determining the maximum derivative of the pulse. For example, the gradient 11 of the first heart pulse sample 202 may be calculated by calculating the derivative of the first heart pulse sample 202 at a number of discrete time intervals or time points between t₁ and t₂ and identifying the maximum derivative calculated (e.g., the maximum derivative of the systolic component). While not shown in FIG. 2, the time point at which the pulse reaches the gradient point—i.e., reaches the maximum derivative or rate of change—may also be recorded. For the second heart pulse sample 204, the gradient 72 may be calculated as the maximum derivative between time points t₃ and t₅.

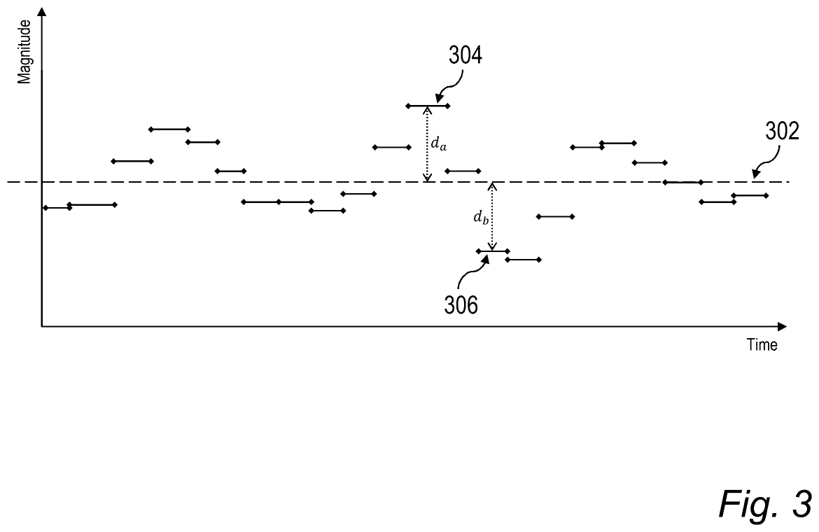

FIG. 3 illustrates steady state baseline wander features that may be extracted from pulse data. FIG. 3 shows a baseline 302 for a sequence of pulse data along with a first representative value 304 for a first heart pulse sample and a second representative value 306 for a second heart pulse sample. Horizontal bars shown in FIG. 3, such as those identified by the first representative value 304 and the second representative value 306, corresponds to a representative value of a heart pulse sample within the pulse data.

The baseline 302 corresponds to the average magnitude of heart pulse samples in the pulse data. The first representative value 304 corresponds to the average magnitude of the first heart pulse sample. The difference dₐ between the first representative value 304 and the baseline 302 represents the baseline wander of the first heart pulse sample. In this instance, dₐ>0. Similarly, the second representative value 306 corresponds to the average magnitude of the second heart pulse sample and the difference d_{b} between the second representative value 306 and the baseline 302 represents the baseline wander of the second heart pulse sample. In this instance, d_{b}<0.

As such, the steady state baseline wander of a pulse may be calculated by determining the difference between a representative magnitude value of the pulse and a baseline magnitude value. The baseline magnitude value may be calculated as the average (e.g., mean or median) magnitude value of all heart pulse samples within the pulse data. Alternatively, the baseline magnitude value may be a fixed value such as 0. The representative magnitude value of a pulse may be a value such as the average (e.g., mean, median, etc.) magnitude value of the pulse or the maximum magnitude value of the pulse. The difference between the representative value of the pulse and the baseline magnitude value therefore indicates the degree to which the pulse has drifted, or wandered, from the baseline. As illustrated in FIG. 3, the sign of the difference between representative values of heart pulse samples and the baseline value varies, or cycles, according to respiratory cycle stage such that the baseline wander value may be used as a feature for encoding heart pulse samples according to respiratory cycle stage.

FIGS. 4A-4E provide an illustrative example of respiratory onset detection from heart pulse data. FIG. 4A shows a chart illustrating pulse data 402 obtained from a wearable physiological monitor for a user over a time period. The skilled person will appreciate that the pulse data 402 shown in FIG. 4A is a representation of a photoplethysmography (PPG) waveform that has been deliberately simplified for ease of reference/understanding. A first point 404 and a second point 406 are shown in FIG. 4A and indicate the boundary (i.e., start and end point) of a single heart pulse sample. Other such points corresponding to the start/end of individual heart pulses are represented in FIG. 4A by the filled white circles. The pulse data 402 comprises a plurality of heart pulse samples such as the heart pulse sample between the first point 404 and the second point 406. Individual heart pulse samples may be extracted from the pulse data by utilizing a peak finding algorithm on the inverted segment of pulse data (e.g., the inverted PPG signal). The peaks identified using the peak finding algorithm are then used to define the boundaries between each pulse within the segment of pulse data (e.g., the first point 404 and the second point 406). The pulse data may then be mapped to a feature space that characterizes individual pulse according to respiratory cycle stage. Estimated respiratory waveforms may be generated from the pulse data mapped to the feature space and used to estimate respiratory onsets of the user. This process is described in detail above in relation to FIG. 1A.

FIG. 4B shows a chart illustrating a respiratory waveform 408 of the user during the same time window as FIG. 4A. The respiratory waveform 408 thus corresponds to the breathing cycle of the user that is associated (e.g., temporally aligned) with the pulse data 402 of the user shown in FIG. 4A. FIG. 4B also shows a plurality of known respiratory onsets corresponding to the start of the respiratory cycles that form the respiratory waveform 408. The known respiratory onsets include a first known respiratory onset 410 and a second known respiratory onset 412. Time points associated with known respiratory onsets are shown in FIG. 4B as hashed circles below the horizontal axis. Also shown in FIG. 4B are a plurality of estimated respiratory onsets calculated from the pulse data 402. The plurality of estimated respiratory onsets include a first estimated respiratory onset 414, a second estimated respiratory onset 416, and a third estimated respiratory onset 418. Time points associated with estimated respiratory onsets are shown as filled white circles on the respiratory waveform 408. The estimated respiratory onsets may be used to extract features for estimating respiratory rate variability (RRV) of the user. For example, the absolute difference (in time) between the first estimated respiratory onset 414 and the second estimated respiratory onset 416 may be used to determine a first respiratory cycle length (RCL) value, RCL₁. The absolute difference (in time) between the second estimated respiratory onset 416 and the third estimated respiratory onset 418 may be used to determine a second RCL value, RCL₂. As described above, the RCL values (e.g., RCL₁, RCL₂, etc.) may be used to calculate an RRV value for the user during the time period. The RRV value can be used to determine a physiological condition associated with the user.

As shown in FIGS. 4C-4E, the plurality of estimated respiratory onsets (e.g., the first estimated respiratory onset 414, the second estimated respiratory onset 416, etc. shown in FIG. 4B) may be determined from estimated respiratory waveforms generated from the pulse data mapped to a feature space that encodes individual pulses according to respiratory cycle stage.

FIG. 4C shows a first feature value 420, a second feature value 422, and an estimated respiratory waveform 424. FIG. 4C also shows a first extrema 426 of the estimated respiratory waveform 424 and a second extrema 428 of the estimated respiratory waveform 424. The first feature value 420 and the second feature value 422 are heart pulse frequency variation features extracted from the pulse data 402 of FIG. 4A at a first and second time point respectively (not shown). Other heart pulse frequency variation feature values extracted from the pulse data 402 of FIG. 4A are shown in FIG. 4C as small filled black circles. The estimated respiratory waveform 424 is generated from the feature values by interpolating between the feature values. The estimated respiratory waveform 424 corresponds to an approximate representation of the user's breathing cycles over the time window spanned by the pulse data 402 of FIG. 4A. Extrema (local maxima) identified from the estimated respiratory waveform 424 may be used to identify respiratory onsets of the user during the time window. FIG. 4C shows two local maxima of the estimated respiratory waveform 424—the first extrema 426 that occurs at time point tₐ and the second extrema 428 that occurs at time point t_{b}. Thus, it can be predicted from the estimated respiratory waveform 424 that the first two respiratory onsets of the user within the time window occur around, or at, time points tₐ and t_{b}.

FIG. 4D shows a third feature value 430, a fourth feature value 432, and an estimated respiratory waveform 434. FIG. 4D also shows a first extrema 436 of the estimated respiratory waveform 434 and a second extrema 438 of the estimated respiratory waveform 434. The third feature value 430 and the fourth feature value 432 are heart pulse amplitude variation features extracted from the pulse data 402 of FIG. 4A at a third and fourth time point respectively (not shown). Other heart pulse amplitude variation feature values extracted from the pulse data 402 of FIG. 4A are shown in FIG. 4D as small filled black circles. The estimated respiratory waveform 434 is generated from the feature values by interpolating between the feature values. The estimated respiratory waveform 434 corresponds to an approximate representation of the user's breathing cycles over the time window spanned by the pulse data 402 of FIG. 4A. Extrema (local maxima) identified from the estimated respiratory waveform 434 may be used to identify respiratory onsets of the user during the time window. FIG. 4D shows two local maxima of the estimated respiratory waveform 434—the first extrema 436 that occurs at time point t_{c} and the second extrema 438 that occurs at time point tₐ. Thus, it can be predicted from the estimated respiratory waveform 434 that the first two respiratory onsets of the user within the time window occur around, or at, time points t_{c} and tₐ.

FIG. 4E shows a fifth feature value 440, a sixth feature value 442, and an estimated respiratory waveform 444. FIG. 4E also shows a first extrema 446 of the estimated respiratory waveform 444 and a second extrema 448 of the estimated respiratory waveform 444. The fifth feature value 440 and the sixth feature value 442 are heart pulse gradient variation features extracted from the pulse data 402 of FIG. 4A at a fifth and sixth time point respectively (not shown). Other heart pulse gradient variation feature values extracted from the pulse data 402 of FIG. 4A are shown in FIG. 4D as small filled black circles. The estimated respiratory waveform 444 is generated from the feature values by interpolating between the feature values. The estimated respiratory waveform 444 corresponds to an approximate representation of the user's breathing cycles over the time window spanned by the pulse data 402 of FIG. 4A. Extrema (local minima) identified from the estimated respiratory waveform 444 may be used to identify respiratory onsets of the user during the time window. FIG. 4E shows two local minima of the estimated respiratory waveform 444—the first extrema 446 that occurs at time point the and the second extrema 448 that occurs at time point t_{f}. Thus, it can be predicted from the estimated respiratory waveform 444 that the first two respiratory onsets of the user within the time window occur around, or at, time points tₑ and t_{f}.

As described in more detail above in relation to FIG. 1A, the local extrema identified may be combined or weighted to identify the respiratory onsets of the user (e.g., the estimated respiratory onsets shown in FIG. 4B). For example, the average respiratory onsets determined for each heart pulse sample may be calculated to aggregate or otherwise combine the respiratory onsets determined from each feature mapping. As a further example, the set of all respiratory onsets determined across all feature mappings may be clustered and the average of each cluster used to identify the estimated respiratory onsets. As a further example, the respiratory onsets may be weighted using a machine learning model.

FIG. 5A shows a machine learning model 502 for weighting respiratory onsets. The machine learning model 502 is a simple recurrent neural network (RNN) that may receive input data 504 and generate a weighting 506. The machine learning model 502 comprises an input layer 508, a simple RNN layer 510, and a dense layer 512. The machine learning model 502 may be used to determine weightings to be applied to respiratory onsets determined using the approaches as described above. In one example, the machine learning model 502 may be used to determine a weighting to be applied to a first respiratory onset estimated from heart pulse frequency variation features and a weighting to be applied to a second respiratory onset estimated from heart pulse amplitude variation features. In this example, the input data 504 comprises 2-features (heart pulse frequency variation and heart pulse amplitude variation) extracted from the previous 10 heart pulses. The skilled person will appreciate that more features and more or fewer previous pulses could be used (e.g., 3 features, 4 features, 20 pulses, 30 pulses, etc.). Continuing the previous example, an architecture that may be used is shown in TABLE I below. The machine learning model 502 predicts a probability value—p—that may be used as a weighting factor for the first respiratory onset and the second respiratory onset. That is, the first respiratory onset, estimated according to heart pulse frequency variation features, may be multiplied by p and the second respiratory onset, estimated according to heart pulse amplitude variation features, may be multiplied by (1-p).

TABLE I

| Layer | Shape | Parameters |
|---|---|---|
| Input | (10, 2) | 0 |
| SimpleRNN | (16) | 3047 |
| Dense | (1) | 17 |

The machine learning model 502 may be trained on a training data set of PPG data and true respiration data (e.g., a data set of approximately 10,000 PPG sequences and accompanying truth respiration data). For each segment or sequence of pulse data in the training data set, the PPG peaks of the pulse data are detected and the pulse length (X1) and pulse amplitude (X2) are extracted for each peak. The extracted features are interpolated to obtain signals S1 and S2 from which the local maxima are identified as predicted respiratory onsets O1 and O2. For each onset, the pulse lengths and amplitudes of the previous 10 pulses are extracted and used as input to the model. The model is trained to estimate the probability of the pulse length being useful. To get the labels (or targets) Y for training the model, estimate the error for each predicted onset in O1 and O2 with respect to the truth is calculated, and assigned a label of Y=1 if the error using pulse length is less than that using pulse amplitude and Y=0 otherwise. The machine learning model 502 may be trained using an Adam optimizer and a binary cross entropy loss. The Adam optimizer may be set with a learning rate of 0.0005, exponential decay rates for the first and second moment estimates of 0.9 and 0.999 respectively, and ε=1×10⁻⁸.

FIG. 5B shows an encoder-decoder network 514 for pulse quality assessment classification. The encoder-decoder network is a variational autoencoder (VAE) comprising an encoder network 516 and a decoder network 518. The encoder network 516 maps an input vector x to a mean vector 520 μ and a standard deviation vector 522 σ. A latent vector 524 z is sampled from the distribution defined by the mean vector 520 μ and the standard deviation vector 522 σ. The latent vector 524 z is a low-dimensional representation of the input vector x. The decoder network 518 generates a reconstructed input vector \hat{x} from the latent vector 524 z. In one embodiment, the encoder-decoder network is a supervised VAE further comprising a fully connected layer 526 and a linear activation function 528. The fully connected layer 526 receives the mean vector 520, the standard deviation vector 522, and multimodal inputs 530.

In general, the encoder-decoder network 514 maps a high-dimensional signal x to a low-dimensional latent space. The high-dimensional signal is a pulse of a segment of pulse data. For example, if a pulse waveform is composed of 128 samples, the high-dimensional signal input to the encoder-decoder network 514 would be x∈ [symbol] ¹²⁸. The latent vector 524 z is sampled from a low-dimensional latent space and provides a compact characterization of the pulse. More particularly, the low-dimensional latent space provides greater separability between pulses having different pulse characteristics; for example, different extents of dicrotic notch. Here, the extent of a dicrotic notch represents the difference in amplitude between the systolic peak and dicrotic notch, and the diastolic peak and dicrotic notch. The latent vector 524 z may thus be considered to encode one or more characteristics of the pulse represented by the input vector x. In one example, the latent vector 524 of a pulse is used as a morphology feature and/or a static feature (i.e., the latent vector 524 corresponds to the one or more latent features).

In one implementation, the encoder network 516 maps from a high-dimensional input vector x∈ [symbol] ¹²⁸ to a low dimensional latent space [symbol] ⁴ such that z∈ [symbol] ⁴. The encoder network 516 comprises an input layer, four convolutional layers, a flatten layer, a dense layer, and three output layers (an output layer for the mean vector, an output layer for the standard deviation vector and an output layer for the sampled latent vector). As shown in FIG. 5B, the output layer for the mean vector (i.e., the mean vector 520) and the output layer for the standard deviation (i.e., the standard deviation vector 522) are both connected to the previous dense layer of the encoder network 516. The sample latent vector (i.e., the latent vector 524) is connected to the output layer for the mean vector and the output layer for the standard deviation vector. The architecture of the encoder network 516 is shown in Table II.

TABLE II

| Layer | Shape | Parameters |
|---|---|---|
| Input | (128, 1) | 0 |
| Gaussian Noise | (128, 1) | 0 |
| Convolutional 1D | (64, 8) | 88 |
| Convolutional 1D | (32, 16) | 1296 |
| Convolutional 1D | (16, 16) | 2576 |
| Convolutional 1D | (8, 16) | 2576 |
| Flatten | 128 | 0 |
| Dense | 16 | 2064 |
| Output (μ) | 4 | 68 |
| Output (σ) | 4 | 68 |
| Output (z) | 4 | 0 |

In the same implementation, the decoder network 518 reconstructs the high-dimensional reconstructed input vector î ∈ [symbol] ¹²⁸ from the low-dimensional latent vector z∈ [symbol] ⁴. The decoder network 518 comprises an input layer, a dense layer, a reshape layer, four 1-dimensional convolutional transpose layers, and a dense layer. The architecture of the decoder network 518 is shown in Table III.

TABLE III

| Layer | Shape | Parameters |
|---|---|---|
| Input | 4 | 0 |
| Dense | 128 | 640 |
| Reshape | (8, 16) | 0 |
| Convolutional 1D Transpose | (16, 16) | 2576 |
| Convolutional 1D Transpose | (32, 16) | 2576 |
| Convolutional 1D Transpose | (64, 16) | 2576 |
| Convolutional 1D Transpose | (128, 8) | 1288 |
| Dense | (128, 1) | 9 |

The encoder-decoder network 514 may be trained on a dataset of over 1 million synthetic PPG pulses and approximately 6,000 annotated real pulses. The dataset of synthetic PPG pulses may be generated according to a mixture of Gaussian model parameterized by the mean of the two gaussians, the standard deviation of the two gaussians, and a multiplicative factor for the amplitude of the first gaussian. In one implementation, the encoder-decoder network 514 is trained over 150 epochs using a minibatch stochastic gradient descent with a binary cross entropy loss function and a Kullback-Liebler divergence regularization term.

As stated above, the encoder-decoder network 514 may be a supervised VAE further comprising the fully connected layer 526 and the linear activation function 528. The fully connected layer 526 receives the latent vector 524 and may receive the multimodal inputs 530. The linear activation function 528 is connected to the fully connected layer 526 to provide a value p∈[−1, +1]. The value p may thus be understood as a degree of “notchiness” of the pulse represented by the input vector (i.e., the extent/depth of the dicrotic notch within the pulse). This value may thus be used to determine whether or not a pulse extracted from a segment of pulse data is a valid or clean pulse. For example, a pulse having a p value greater than 0 may be determined as a valid pulse and thus used to determine blood pressure while a pulse having p value less than or equal to 0 may be discarded from further processing as an invalid or noisy pulse. Additionally, or alternatively, a pulse is also assessed based on the autoencoder's reconstruction loss; if the loss value is above a threshold value, then the pulse is determined to be possibly affected by noise and is hence excluded from further processing.

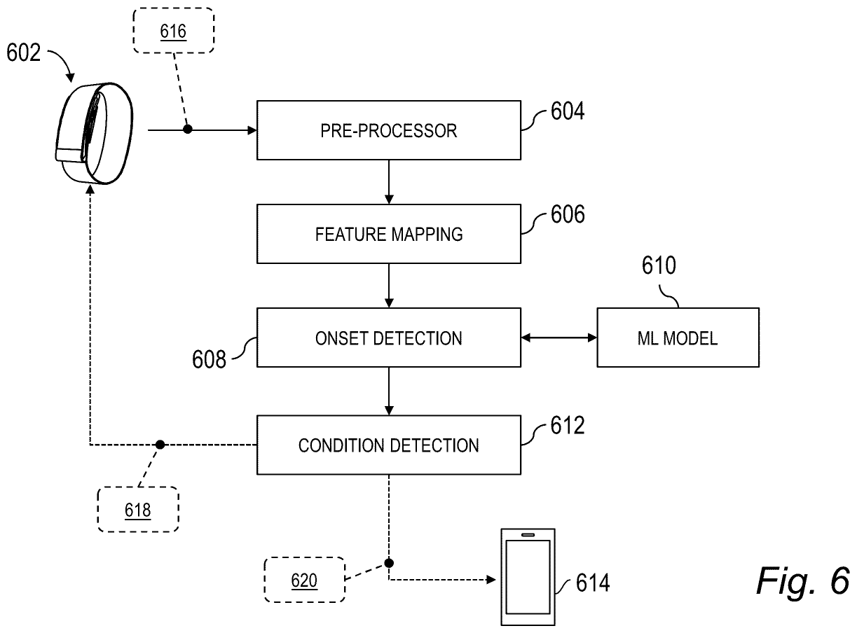

FIG. 6 shows a portion of a system for respiratory rate onset estimation and physiological condition detection. FIG. 6 shows a wearable physiological monitor 602, a pre-processor module 604, a feature mapping module 606, an onset detection module 608, a machine learning model 610, and a condition detection module 612. FIG. 6 also shows a device 614, pulse data 616, a first notification 618, and a second notification 620.

The wearable physiological monitor 602 may be any suitable physiological monitor that can obtain pulse data from a user (e.g., a user wearing the physiological monitor). For example, the physiological monitor 602 may be the physiological monitor 1006 shown in FIG. 10. Similarly, the device 614 may be any suitable device such as the one or more devices 1020 shown in FIG. 10. The operations performed by the modules shown in FIG. 6 (e.g., the pre-processor module 604, the feature mapping module 606, the onset detection module 608, the machine learning model 610, and/or the condition detection module 612) may be performed by one or more of the wearable physiological monitor 602, the device 614, and/or a remote computing device or server (not shown).

The pulse data 616 obtained from the wearable physiological monitor 602 represents the cardiac activity of the user. For example, the pulse data 616 may be a segment of pulse data related to cardiac activity of the user during a time period/window such as a portion of a sleep session. The pulse data 616 comprises a waveform or time-series of values that characterize the cardiac activity of the user during the time window and so comprises one or more pulses appearing as peaks within the pulse data 616. In one example, the pulse data 616 comprises between 20 and 40 pulses (i.e., heart beats). Individual pulses within the pulse data 616 may be extracted or identified using standard approaches (e.g., peak finding on the inverse pulse data signal as described above).

The pulse data 616 obtained by the wearable physiological monitor 602 may be pre-processed by the pre-processor module 604 before being provided to the feature mapping module 606. The pre-processing may include applying one or more filters to the pulse data 616 (e.g., a low-pass filter, a high-pass filter, a moving average filter, etc.), normalizing the pulse data 616, detrending the pulse data 616, and/or performing quality assessment of the pulse data 616.

The feature mapping module 606 may be used to map the pulse data 616 to a feature space that characterizes/encodes pulses according to respiratory cycle stage. For example, pulses occurring towards the start of a respiratory cycle may be grouped in one region of the feature space, pulses occurring towards the middle of a respiratory cycle may be grouped in another region of the feature space, and pulses occurring towards the end of a respiratory cycle may be grouped in yet another region of the feature space. The feature mapping module 606 may utilize a feature transformation approach to map the pulse data 616 to a feature space. For example, a neural network (e.g., an autoencoder, a variational autoencoder, etc.) or a manifold learning algorithm (e.g., Principal Components Analysis, Isomap, UMAP, etc.) may be used to transform the pulse data 616 into a lower-dimensional or latent feature space. Alternatively, the feature mapping module 606 may utilize a feature extraction approach to map the pulse data 616 to a feature space whereby the extracted features characterize the modulation, or change, of heart pulse samples with respect to respiratory cycle (e.g., a heart pulse frequency variation feature, a heart pulse amplitude variation feature, a heart pulse gradient variation feature, etc.).

The pulse data 616 mapped to the feature space may be used to approximate the respiratory waveform of the user during the time window. That is, because the feature mapping module 606 maps the pulse data 616 to a feature space that characterizes pulses according to respiratory cycle stage, the heart pulse samples are mapped from heart rate, or pulse, space to respiratory, or breathing, space. That is, heart pulses of a user exhibit different morphological and temporal characteristics at different time points of a respiratory cycle of the user; the features extracted from a heart pulse quantify, or characterize, these morphological characteristics thereby enabling respiratory data to be approximated from heart pulse data without requiring additional equipment or the use of invasive techniques. The onset detection module 608 generates one or more estimated respiratory waveforms from the pulse data 616 mapped to the feature space. For example, the onset detection module 608 may interpolate the pulse data 616 mapped to the feature space to generate a signal or waveform that is indicative of the respiratory cycle, or breathing pattern/cycle, of the user during the time window. The onset detection module 608 may then identify local extrema of these signals or waveforms to identify respiratory onsets of the user during the time window. The onset detection module 608 may use a weighting strategy to combine respiratory onsets determined from different modulatory features. For example, the onset detection module 608 may use the machine learning model 610 (e.g., the machine learning model 502 shown in FIG. 5A) to determine a weighting for respiratory onsets determined using heart pulse frequency and heart pulse amplitude features.

The condition detection module 612 may use the respiratory onsets determined by the onset detection module 608 to detect or identify a physiological condition associated with the user (e.g., recovery rate, sleep stage, etc.). The condition detection module 612 may calculate a respiratory rate variability (RRV) score based on a metric calculated using the respiratory onsets. As described in more detail above in relation to FIG. 1B, the metric may utilize changes in respiratory cycle length across the time window to calculate the RRV score for the user. The condition detection module 612 may cause one or more notifications to be generated and output based on the physiological condition and/or the RRV score. The first notification 618 shown in FIG. 6 may be a haptic feedback notification that causes the wearable physiological monitor 602 to output haptic feedback to the user (e.g., based on the RRV score satisfying a threshold condition). The second notification 620 may be displayed by the device 614 thereby notifying the user as to the detected physiological condition and/or RRV scores. The condition detection module 612 may also utilize historical (previous) RRV scores of the user to identify one or more trends or changes in the user's RRV score over time (as described in more detail above in relation to FIG. 1B).

FIG. 7 is a flow chart illustrating a method 700 for respiratory onset estimation based on accelerometer data of a wearable physiological monitor. The method 700 may be used in cooperation with any of the devices, systems, and methods described herein, such as by a user device (e.g., a mobile device) that is communicatively coupled to a wearable, continuous physiological monitoring device. For example, the method 700 may be used with any of the servers, wearable monitors, and other devices described herein. In general, the method 700 calculates respiratory onsets (i.e., the onset of breathing cycles or the start of inhalation) for a user based on accelerometer data obtained from a wearable physiological monitor worn by the user during a period of sleep. Analyzing the movement of the wearable physiological monitor in a principal movement direction—e.g., a direction that is substantially aligned with the direction of gravity-allows the breathing pattern or respiratory cycles of the user to be recovered and used to determine respiratory onsets. Information or data pertaining to the respiratory cycles (or breathing cycles) of a user may be useful for diagnosis and monitoring of respiratory conditions, early detection of respiratory distress, and/or personalized care (e.g., helping aid user recovery, improve sleep, etc.). The method 700 provides a non-invasive technique for recovering information regarding a user's respiratory cycles from data that can be non-invasively obtained from a user's personal device, such as a wearable physiological device worn by the user. The respiratory onsets determined using the method 700 may be incorporated with the respiratory onsets determined using the pulse based analysis described above to provide an accurate multi-modal approach to respiratory onset detection using a user's personal device (e.g., wearable physiological monitor). As a significant advantage, this facilitates an analysis that uses motion data during periods of motion stability and pulse data during periods of cardiac stability, which may, for example, include the same sleep stages or different sleep stages during an interval of sleep for a user. This opens up a rich vein of data to be generated from which insights into the user's physiological condition may be efficiently and effectively obtained. The user is thus provided with “at home” insights into their respiratory state and/or physiological condition without requiring specialized medical equipment or the performance of invasive techniques.

As shown in step 702, the method 700 may include receiving, from a wearable physiological monitor worn by a user, accelerometer data indicative of movement of the wearable physiological monitor during a time window. More specifically, the wearable physiological monitor may be worn on a wrist of the user during the time window. Alternatively, the wearable physiological monitor may be worn on the chest of the user during the time window. Here, the time window corresponds to a portion of a sleep session of the user. The time window may be identified by identifying a period of low motion of the wearable physiological monitor during the time sleep session. Advantageously, focusing analysis on low motion regions helps ensure that the accelerometer data is not affected by motion noise that occurs due to the user's sleeping position or movement. That is, the movement of the wearable physiological monitor during a low motion period is primarily related to motion occurring due to the breathing activity of the user. Moreover, this allows for the method to work across a range of different sleep orientations of the user (e.g., sleeping with hands flat, sleeping with hands perpendicular, sleeping with hands on belly, sleeping on belly with hands flat, etc.).

A period of low motion may be identified by identifying a time period wherein an average motion of the wearable physiological monitor during the time period is below a threshold level of motion. For example, low motion regions may be identified by calculating the standard deviation across a moving window of 2,000 samples (20 packets) with a stride of one sample and looking for regions where the standard deviation drops below a threshold value (e.g., 0.02, 0.01, etc.). These regions are then annotated as the starting point of a low motion region. The point at which the standard deviation subsequently increases above the threshold value is annotated as the ending point of the low motion region. In this way, multiple potential low motion regions may be identified from accelerometer data of a single sleep session. The potential low motion regions may be further filtered by only maintaining regions that span at least 3,000 samples (300 packets).

As shown in step 704, the method 700 may include identifying, based on the accelerometer data, a principal direction of movement of the wearable physiological monitor during the time window. In general, the principal direction of movement of the wearable physiological monitor corresponds to the movement in the direction of gravity as it is the movement in this direction that most faithfully captures the user's breathing patterns. However, due to the sleeping position of the user, the principal direction of movement may not be in a single axis of the accelerometer data (e.g., the Z-axis). Therefore, the principal direction of movement may need to be extracted, or identified, from the accelerometer data.

The principal direction of movement may correspond to a component of maximum variance determined from the accelerometer data. That is, principal components analysis (PCA) may be applied to the accelerometer data and the principal component of maximum variance identified as the principal direction of movement of the wearable physiological monitor. Alternatively, the principal direction of movement may correspond to an axis of movement of the wearable physiological monitor having maximum mean absolute magnitude during the time window. That is, the principal direction of movement may correspond to one of the fixed axes of movement of the accelerometer (e.g., the X-axis, Y-axis, or Z-axis) that has maximum mean absolute magnitude during the time window. Alternatively, the principal direction of movement may be calculated as a dot product of representative accelerometer values along a plurality of axis of the accelerometer. That is, the mean value of the accelerometer for each of the three fixed axes (X, Y, Z) of the accelerometer is calculated and the dot product taken.

As shown in step 706, the method 700 may include generating a first waveform from the accelerometer data. The first waveform comprises data indicative of movement of the wearable physiological monitor along the principal direction of movement. If the principal direction of movement corresponds to one of the fixed axes of the accelerometer (e.g., the X-axis, Y-axis, or Z-axis), then the first waveform corresponds to the 1-dimensional accelerometer signal along that fixed axis. If the principal direction of movement is a component of the accelerometer data (e.g., a principal component), then the accelerometer data may be projected onto the component to generate a 1-dimensional signal comprising data indicative of movement of the wearable physiological monitor along the principal direction of movement. The accelerometer data, or the projection of the accelerometer data, may be interpolated to generate the first waveform (e.g., using cubic spline interpolation).

As shown in step 708, the method 700 may include calculating a plurality of respiratory onsets for the user during the time window based on a plurality of local extrema of the first waveform. More specifically, the first waveform may be inverted and a peak finding algorithm used to identify the peaks (local extrema/maxima) of the inverted waveform that correspond to the locations of inspiration (i.e., respiratory onsets). The time points associated with the local extrema thus correspond to the time points associated with the respiratory onsets of the user. The plurality of respiratory onsets may then be used as part of any of the above described methods to help determine a user's respiratory rate variability and associated physiological condition(s).

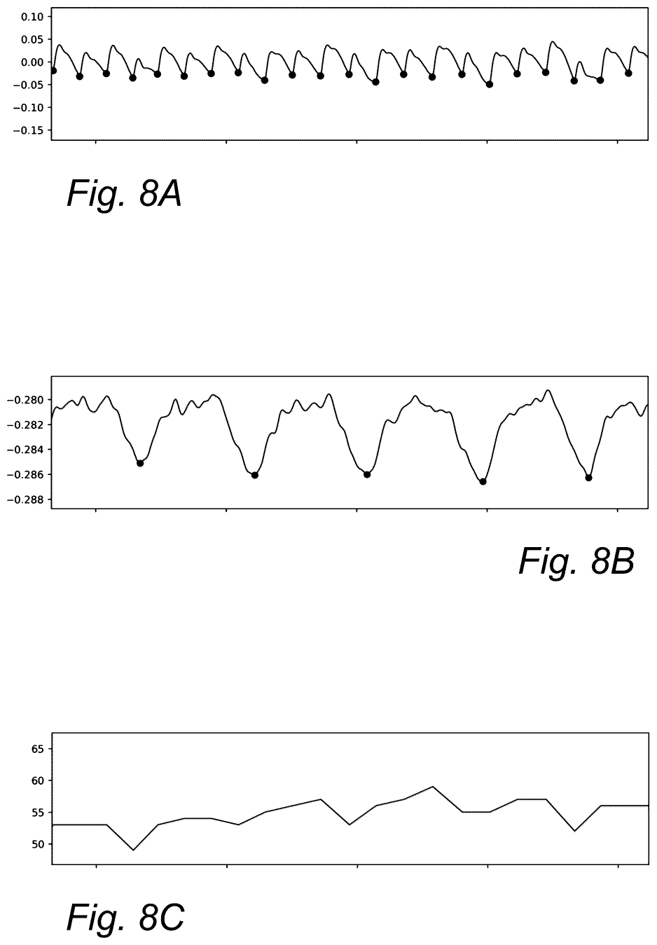

FIGS. 8A-8C illustrate accelerometer based respiratory onset detection. FIG. 8A shows a waveform corresponding to the pulse data of a user during a portion of a sleep session. The filled black circles shown in FIG. 8A correspond to boundary points (e.g., start and end points) of individual heart pulse samples or heartbeats. FIG. 8B shows a respiratory cycle waveform recovered from accelerometer data of a wearable physiological monitor using the method 700 shown in FIG. 7. The filled black circles shown in FIG. 8B correspond to the respiratory onsets identified from the waveform. FIG. 8C shows the ground truth R-R intervals obtained over the portion of the sleep session. It is known that the R-R interval reduces with inspiration and increases with exhalation. As can be seen in the comparison between FIGS. 8B and 8C, the respiratory onsets identified from the accelerometer data substantially aligns temporally with the local minima of the R-R interval data (i.e., the inhalation points).

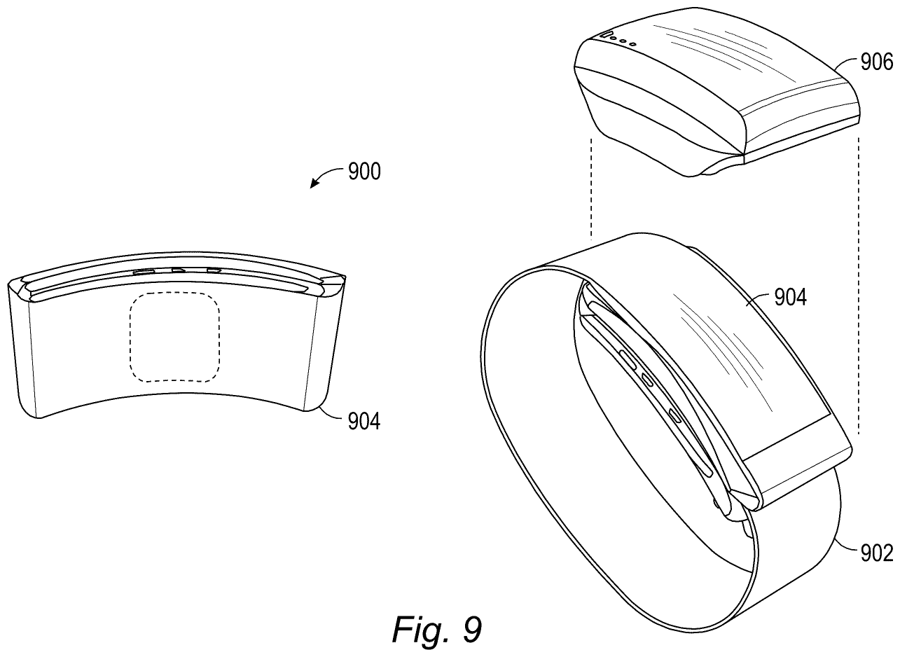

FIG. 9 shows a physiological monitoring device. The overall system 900 may include a device 904 (that may or may not include a display screen or other user interface) generally configured for physiological monitoring. The system 900 may further include a removable and replaceable battery 906 for recharging the device 904. A strap 902 may be provided and may include any arrangement suitable for retaining the device 904 in a position on a wearer's body for acquisition of physiological data as described herein. For example, the strap 902 may include slim elastic band formed of any suitable elastic material, for example, a rubber, a woven polymer fiber such as a woven polyester, polypropylene, nylon, spandex, and so forth. The strap 902 may be adjustable to accommodate different wrist sizes, and may include any latches, hasps, or the like to secure the device 904 in an intended position for monitoring a physiological signal. While a wrist-worn device is depicted, it will be understood that the device 904 may be configured for positioning in any suitable location on a user's body, based on the sensing modality and the nature of the signal to be acquired. For example, the device 904 may be configured for use on a wrist, an ankle, a bicep, a chest, or any other suitable location(s), and the strap 902 may be, or may include, a waistband or other elastic band or the like within an article of clothing or accessory. The device 904 may also or instead be structurally configured for placement on or within a garment, e.g., permanently or in a removable and replaceable manner. To that end, the device 904 may be structurally configured for placement within a pocket, slot, and/or other housing that is coupled to or embedded within a garment. In such configurations, the garment may include sensing windows or other pathways such that the device 904 can sense physiological and/or biomechanical parameters from a user wearing a garment that includes the device 904 therein or thereon.

The system 900 may include any hardware components, subsystems, and the like to provide various functions such as data collection, processing, display, and communications with external resources. For example, the system 900 may include a heart rate monitor using, e.g., photoplethysmography, electrocardiogram any other technique(s). The system 900 may be configured such that, when placed for use about a wrist, the system 900 initiates acquisition of physiological data from the wearer. In some embodiments, the pulse or heart rate may be taken using an optical sensor coupled with one or more light emitting diodes (LEDs), all directly in contact with the user's wrist. The LEDs may be positioned to direct illumination toward the user's skin and may be accompanied by one or more photodiodes or other photodetectors suitable for measuring illumination from the LEDs that is reflected and/or transmitted by the wearer's skin.

The system 900 may be configured to record other physiological and/or biomechanical parameters including, but not limited to, skin temperature (using a thermometer), galvanic skin response (using a galvanic skin response sensor), motion (using one or more multi-axes accelerometers and/or gyroscope), blood pressure, and the like, as well environmental or contextual parameters such as ambient light, ambient temperature, humidity, time of day, and the like. The system 900 may also include other sensors such as accelerometers and/or gyroscopes for motion detection, and sensors for environmental temperature sensing, electrodermal activity (EDA) sensing, galvanic skin response (GSR) sensing, and the like.

The system 900 may include one or more sources of battery life, such as a first battery environmentally sealed within the device 904 and a battery 906 that is removable and replaceable to recharge the battery in the device 904. Also or instead, the system 900 may include a plurality of devices 904, where such devices 904 may be able to provide power to one another. The system 900 may perform numerous functions related to continuous monitoring, such as automatically detecting when the user is asleep, awake, exercising, and so forth, and such detections may be performed locally at the device 904 or at a remote service coupled in a communicating relationship with the device 904 and receiving data therefrom. In general, the system 900 may support continuous, independent monitoring of a physiological signal such as a heart rate, and acquired data may be stored on the device 904 until it can be uploaded to a remote processing resource for more computationally expensive analysis.

FIG. 10 illustrates a physiological monitoring system. More specifically, FIG. 10 illustrates a physiological monitoring system 1000 that may be used with any of the methods or devices described herein. In general, the system 1000 may include a physiological monitor 1006, a user device 1020, a remote server 1030 with a remote data processing resource (such as any of the processors or processing resources described herein), and one or more other resources 1050, all of which may be interconnected through a data network 1002.

The data network 1002 may be any of the data networks described herein. For example, the data network 1002 may be any network(s) or internetwork(s) suitable for communicating data and information among participants in the system 1000. This may include public networks such as the Internet, private networks, telecommunications networks such as the Public Switched Telephone Network or cellular networks using third generation (e.g., 3G or IMT-200), fourth generation (e.g., LTE (E-UTRA) or WiMAX-Advanced (IEEE 1002.16 m)), fifth generation (e.g., 5G), and/or other technologies, as well as any of a variety of corporate area or local area networks and other switches, routers, hubs, gateways, and the like that might be used to carry data among participants in the system 1000. This may also include local or short range communications networks suitable, e.g., for coupling the physiological monitor 1006 to the user device 1020, or otherwise communicating with local resources.

The physiological monitor 1006 may, in general, be any physiological monitoring device, such as any of the wearable monitors or other monitoring devices described herein. Thus, the physiological monitor 1006 may generally be shaped and sized to be worn on a wrist or other body location and retained in a desired orientation relative to the appendage with a strap 1010 or other attachment mechanism. The physiological monitor 1006 may include a wearable housing 1011, a network interface 1012, one or more sensors 1014, one or more light sources 1015, a processor 1016, a haptic device 1017 (and/or any other type of component suitable for providing haptic or other sensory alerts to a user), a memory 1018, and a wearable strap 1010 for retaining the physiological monitor 1006 in a desired location on a user.

In general, the physiological monitor 1006 may include a wearable physiological monitor configured to acquire heart rate data and/or other physiological data from a wearer. More specifically, the wearable housing 1011 of the physiological monitor 1006 may be configured such that a user can acquire heart rate data and/or other physiological data from the user in a substantially continuous manner. The wearable housing 1011 may be configured for cooperation with a strap 1010 or the like, e.g., for engagement with an appendage of a user.

The network interface 1012 may be configured to coupled one or more participants of the system 1000 in a communicating relationship, e.g., with the remote server 1030, either directly, e.g., through a cellular data connection or the like, or indirectly through a short range wireless communications channel coupling the physiological monitor 1006 locally to a wireless access point, router, computer, laptop, tablet, cellular phone, or other device that can relay data from the physiological monitor 1006 to the remote server 1030 as necessary or helpful for acquiring and processing data.

The one or more sensors 1014 may include any of the sensors described herein, or any other sensors suitable for physiological monitoring. By way of example and not limitation, the one or more sensors 1014 may include one or more of a light source, an optical sensor, an accelerometer, a gyroscope, a temperature sensor, a galvanic skin response sensor, a capacitive sensor, a resistive sensor, an environmental sensor (e.g., for measuring ambient temperature, humidity, lighting, and the like), a geolocation sensor, a temporal sensor, an electrodermal activity sensor, and the like. The one or more sensors 1014 may be disposed in the wearable housing 1011 or otherwise positioned and configured for capture of data for physiological monitoring of a user. In one aspect, the one or more sensors 1014 include a light detector configured to provide data to the processor 1016 for calculating a heart rate variability. The one or more sensors 1014 may also or instead include an accelerometer configured to provide data to the processor 1016, e.g., for detecting activities such as a sleep state, a resting state, a waking event, exercise, and/or other user activity. In an implementation, the one or more sensors 1014 measure a galvanic skin response of the user.

The processor 1016 and memory 1018 may be any of the processors and memories described herein and may be suitable for deployment in a physiological monitoring device. In one aspect, the memory 1018 may store physiological data obtained by monitoring a user with the one or more sensors 1014. The processor 1016 may be configured to obtain heart rate data from the user based on the data from the sensors 1014. The processor 1016 may be further configured to assist in a determination of a condition of the user, such as whether the user has an infection or other condition of interest as described herein.

The one or more light sources 1015 may be coupled to the wearable housing 1011 and controlled by the processor 1016. At least one of the light sources 1015 may be directed toward the skin of a user's appendage. Light from the light source 1015 may be detected by the one or more sensors 1014.

The system 1000 may further include a remote data processing resource executing on a remote server 1030. The remote data processing resource may be any of the processors described herein, and may be configured to receive data transmitted from the memory 1018 of the physiological monitor 1006, and to process the data to detect or infer physiological signals of interest such as heart rate, heart rate variability, respiratory rate, pulse oxygen, blood pressure, and so forth. The remote server 1030 may also or instead evaluate a condition of the user such as a recovery state, sleep quality, daily activity strain, and any health conditions that might be detected based on such data.

The system 1000 may also include one or more user devices 1020, which may work together with the physiological monitor 1006, e.g., to provide a display for user data and analysis, and/or to provide a communications bridge from the network interface 1012 of the physiological monitor 1006 to the data network 1002 and the remote server 1030. For example, physiological monitor 1006 may communicate locally with a user device 1020, such as a smartphone of a user, via short-range communications, e.g., Bluetooth, or the like, e.g., for the exchange of data between the physiological monitor 1006 and the user device 1020, and the user device 1020 may communicate with the remote server 1030 via the data network 1002. Computationally intensive processing, such as infection monitoring, may be performed at the remote server 1030, which may have greater memory capabilities and processing power than the physiological monitor 1006 that acquires the data.

The user device 1020 may include any computing device as described herein, including without limitation a smartphone, a desktop computer, a laptop computer, a network computer, a tablet, a mobile device, a portable digital assistant, a cellular phone, a portable media or entertainment device, and so on. The user device 1020 may provide a user interface 1022 for access to data and analysis by a user, and/or to control operation of the physiological monitor 1006. The user interface 1022 may be maintained by a locally executing application on the user device 1020, or the user interface 1022 may be remotely served and presented on the user device 1020, e.g., from the remote server 1030 or the one or more other resources 1050.

In general, the remote server 1030 may include data storage, a network interface, and/or other processing circuitry. The remote server 1030 may process data from the physiological monitor 1006 and perform infection monitoring/analyses or any of the other analyses described herein and may host a user interface for remote access to this data, e.g., from the user device 1020. The remote server 1030 may include a web server or other programmatic front end that facilitates web-based access by the user devices 1020 or the physiological monitor 1006 to the capabilities of the remote server 1030 or other components of the system 1000.

The other resources 1050 may include any resources that can be usefully employed in the devices, systems, and methods described herein. For example, these other resources 1050 may include without limitation other data networks, human actors (e.g., programmers, researchers, annotators, editors, analysts, and so forth), sensors (e.g., audio or visual sensors), data mining tools, computational tools, data monitoring tools, algorithms, and so forth. The other resources 1050 may also or instead include any other software or hardware resources that may be usefully employed in the networked applications as contemplated herein. For example, the other resources 1050 may include payment processing servers or platforms used to authorize payment for access, content, or option/feature purchases, or otherwise. In another aspect, the other resources 1050 may include certificate servers or other security resources for third-party verification of identity, encryption or decryption of data, and so forth. In another aspect, the other resources 1050 may include a desktop computer or the like co-located (e.g., on the same local area network with, or directly coupled to through a serial or USB cable) with a user device 1020, wearable strap 1010, or remote server 1030. In this case, the other resources 1050 may provide supplemental functions for components of the system 1000.

The other resources 1050 may also or instead include one or more web servers that provide web-based access to and from any of the other participants in the system 1000. While depicted as a separate network entity, it will be readily appreciated that the other resources 1050 (e.g., a web server) may also or instead be logically and/or physically associated with one of the other devices described herein, and may for example, include or provide a user interface 1022 for web access to a remote server 1030 or a database in a manner that permits user interaction through the data network 1002, e.g., from the physiological monitor 1006 or the user device 1020, with processing and data resources of the remote server 1030.

One limitation on wearable sensors can be body placement. Devices are typically wrist-based and may occupy a location that a user would prefer to reserve for other devices or jewelry, or that a user would prefer to leave unadorned for aesthetic or functional reasons. This location also places constraints on what measurements can be taken and may also limit user activities. For example, a user may be prevented from wearing boxing gloves while wearing a sensing device on their wrist. To address this issues, physiological monitors may also or instead be embedded in clothing, which may be specifically adapted for physiological monitoring with the addition of communications interfaces, power supplies, device location sensors, environmental sensors, geolocation hardware, payment processing systems, and any other components to provide infrastructure and augmentation for wearable physiological monitors. Such “smart garments” offer additional space on a user's body for supporting monitoring hardware and may further enable sensing techniques that cannot be achieved with single sensing devices. For example, embedding a plurality of physiological sensors or other electronic/communication devices in a shirt may allow electrocardiogram (ECG) based heart rate measurements to be gathered from a torso region of the wearer; wireless antennas to be placed above the upper portion of the thoracic spine to achieve desired communications signals; a contactless payment system to be embedded in a sleeve cuff for interactions with a payment terminal; and muscle oxygen saturation measurements to be gathered from muscles such as the pectoralis major, latissimus dorsi, biceps brachii, and other major muscle groups. This non-exhaustive list illustrates just some examples of technology that may be incorporated into a single garment.

Smart garments may also free up body surfaces for other devices. For example, if sensors in a wrist-worn device that provide heart rate monitoring and step counting can be instead embedded in a user's undergarments, the user may still receive the biometric information they desire, while also being able to wear jewelry or other accessories for suitable occasions.

The present disclosure generally includes smart garment systems and techniques. It will be understood that a “smart garment” as described herein generally includes a garment the incorporates infrastructure and devices to support, augment, or complement various physiological monitoring modes. Such a garment may include a wired, local communication bus for intra-garment hardware communications, a wireless communication system for intra-garment hardware communications, a wireless communication system for extra-garment communications and so forth. The garment may also or instead include a power supply, a power management system, processing hardware, data storage, and so forth, any of which may support enriched functions for the smart garment.

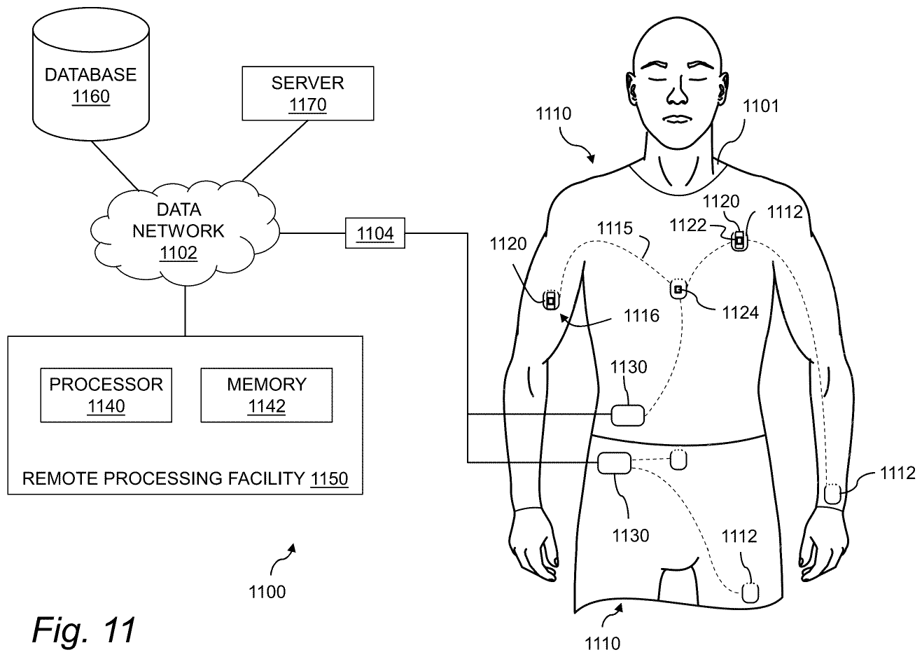

FIG. 11 shows a smart garment system. In general, the system 1100 may include a plurality of components—e.g., a garment 1110, one or more modules 1120, a controller 1130, a processor 1140, a memory 1142, and so on—capable of communicating with one another over a data network 1102. The garment 1110 may be wearable by a user 1101 and configured to communicate with a module 1120 having a physiological sensor 1122 that is structurally configured to sense a physiological parameter of the user 1101. As discussed herein, the module 1120 may be controllable by the controller 1130 based at least in part on a location 1116 where the module 1120 is located on or within the garment 1110. This position-based information may be derived from an interaction and/or communication between the module 1120 and the garment 1110 using various techniques. It will be understood that, while two controllers 1130 are shown, the garment 1110 may include a single inter-garment controller, or any number of separate controllers 1130 in any number of garments 1110 (e.g., one per garment, or one for all garments worn by a person, etc.), and/or controllers may be integrated into other modules 1120.

For communication over the data network 1102, the system 1100 may include a network interface 1104, which may be integrated into the garment 1110, included in the controller 1130, or in some other module or component of the system 1100, or some combination of these. The network interface 1104 may generally include any combination of hardware and software configured to wirelessly communicate data to remote resources. For example, the network interface 1104 may use a local connection to a laptop, smart phone, or the like that couples, in turn, to a wide area network for accessing, e.g., web-based or other network-accessible resources. The network interface 1104 may also or instead be configured to couple to a local access point such as a router or wireless access point for connecting to the data network 1102. In another aspect, the network interface 1104 may be a cellular communications data connection for direct, wireless connection to a cellular network or the like.

The data network 1102 may generally include any communication network through which computer systems may exchange data. For example, the data network 1102 may include, but is not limited to, the Internet, an intranet, a LAN (Local Area Network), a WAN (Wide Area Network), a MAN (Metropolitan Area Network), a wireless network, a cellular data network, an optical network, and the like. To exchange data via the data network 1102, the system 1100 and the data network 1102 may use various methods, protocols, and standards including, but not limited to, token ring, Ethernet, wireless Ethernet, Bluetooth, TCP/IP, UDP, HTTP, FTP, SNMP, SMS, MMS, SS7, JSON, XML, REST, SOAP, CORBA, IIOP, RMI, DCOM and Web Services. To ensure data transfer is secure, the system 1100 may transmit data via the data network 1102 using a variety of security measures including, but not limited to, TSL, SSL and VPN. By way of example, some embodiments of the system 1100 may be configured to stream information wirelessly to a social network, a data center, a cloud service, and so forth.

In some embodiments, data streamed from the system 1100 to the data network 1102 may be accessed by the user 1101 (or other users) via a website. The network interface 1104 may thus be configured such that data collected by the system 1100 is streamed wirelessly to a remote processing facility 1150, database 1160, and/or server 1170 for processing and access by the user. In some embodiments, data may be transmitted automatically, without user interactions, for example by storing data locally and transmitting the data over available local area network resources when a local access point such as a wireless access point or a relay device (such as a laptop, tablet, or smart phone) is available. In some embodiments, the system 1100 may include a cellular system or other hardware for independently accessing network resources from the garment 1110 without requiring local network connectivity.

In one example, the network interface 1104 may be configured to stream data using Bluetooth or Bluetooth Low Energy technology, e.g., to a nearby device such as a cell phone or tablet for forwarding to other resources on the data network 1102. In another example, the network interface 1104 may be configured to stream data using a cellular data service, such as via a 3G, 4G, or 5G cellular network. It will be understood that the network interface 1104 may include a computing device such as a mobile phone or the like. The network interface 1104 may also or instead include or be included on another component of the system 1100, or some combination of these. Where battery power or communications resources can advantageously be conserved, the system 1100 may preferentially use local networking resources when available, and reserve cellular communications for situations where a data storage capacity of the garment 1110 is reaching capacity. Thus, for example, the garment 1110 may store data locally up to some predetermined threshold for local data storage, below which data is transmitted over local networks when available. The garment 1110 may also transmit data to a central resource using a cellular data network only when local storage of data exceeds the predetermined threshold.

The garment 1110 may include one or more of a shirt (or other top), shorts/pants (or other bottom), an undergarment (e.g., undershirt, underwear, brassiere, and so on), a sock or other footwear, a shoe, a facemask, a hat or helmet (or other head adornment), a compression sleeve, a sweatband, kinesiology tape or elastic therapeutic tape, a glove, and the like. More generally, the garment 1110 may include any type(s) of wearable clothing or adornment suitable for wearing by a user and retaining one or more sensing modules as contemplated herein.

The garment 1110 may include one or more designated areas 1112 for positioning a module to sense a physiological parameter of the user 1101 wearing the garment 1110. One or more of the designated areas 1112 may be specifically tailored for receiving a module 1120 therein or thereon. For example, a designated area 1112 may include a pocket structurally configured to receive a module 1120 therein. Also or instead, a designated area 1112 may include a first fastener configured to cooperate with a second fastener disposed on a module 1120. One or more of the first fastener and the second fastener may include at least one of a hook-and-loop fastener, a button, a clamp, a clip, a snap, a projection, and a void.

The designated areas 1112 may include at least one of a torso region, a spinal region, an extremity region (e.g., one or more of an arm region such as a sleeve, and a leg region such as a pant leg), a waistband region, a cuff region, and so on. Also or instead, one or more of the designated areas 1112 may include at least a region adjacent to one or more muscle groups of the user 1101, e.g., muscle groups including at least one of the pectoralis major, latissimus dorsi, biceps brachii, and so on.

By placing a pocket or the like in one of these designated areas 1112, a position of a module 1120 can be controlled, and where an RFID tag, sensor, or the like is used, the designated area 1112 can specifically sense when a module 1120 is positioned there for monitoring, and can communicate the detected location to any suitable control circuitry. In this manner, a garment 1110 may facilitate the installation of modules 1120 in many different, discrete locations, the placement of which can be controlled by the configuration of the garment 1110, and the use of which can be automatically detected when corresponding control modules 1120 are placed there for use. Also or instead, the garment 1110 may facilitate the placing of the modules 1120 over relatively large regions of the garment 1110. For example, a garment 1110 may include a relatively large region (in terms of surface area) where a module 1120 can be affixed or otherwise secured, e.g., by loops, straps, buttons, sheets of hook-and-loop fasteners, and so forth.

In general, each designated area 1112 may include a pocket such as any of those described above, or any other mounting fixture or combination of fixtures. Where a pocket is used, the pocket may be configured as described above to preferentially urge a module 1120 within the pocket toward the user's skin under normal pressure. Without limiting the generality of the foregoing, this may generally include an exterior layer of the pocket that is less elastic than an interior surface of the pocket so that when circumferential tension is applied (e.g., when the garment 1110 is donned), the pocket preferentially urges a contact surface of the sensor inward toward the intended target surface with at least a predetermined normal force (when the garment 1110 is properly sized for the user). In this respect, it will be understood that although some variation in normal force among users and garments is inevitable, typical tensions for comfortable use of properly fitted athletic wear are generally known, and adequate contact force to obtain a high quality physiological signal is generally known, and in any event readily observable in acquired data. As such, adequate circumferential tensions and resulting normal contact forces needed to promote good contact between sensing regions of the module 1120 (such as LEDs, capacitive touch sensors, photodiodes, and the like) and the user's skin may readily be determined, and can advantageously facilitate the use of wrist-worn sensor housings such as those described above with one of the garments 1110 described herein for off-wrist monitoring if/when desired.

In one aspect, the designated areas 1112 may usefully be positioned where reinforcing elastic bands are typically provided on garments, e.g., around the mid-torso for a sports bra, around the waist on shorts or underwear, or on the sleeves of a t-shirt. In one aspect, the designated areas 1112 may also usefully be positioned according to the intended physiological measurement, e.g., near major arteries suitable for heart rate detection using photoplethysmography. In one aspect, the garment 1110 may usefully distribute these designated areas 1112 (and supporting infrastructure such as wired connectors, location identification tags, and the like) at the intersection of regions where good physiological signals can be obtained and regions where adequate normal forces for good sensor contact can be generated by clothing. For example, this may include the ankles, the waist, the mid-torso, the biceps, the wrists, the forehead, and so on.

The garment 1110 may also or instead incorporate other infrastructure 1115 to cooperate with a module 1120. For example, the garment infrastructure 1115 may include wires or the like embedded in the garment 1110 to facilitate wired data or power transfer between installed modules 1120 and other system components (including other modules 1120). The infrastructure 1115 may also or instead include integrated features for, e.g., powering modules, supporting data communications among modules, and otherwise supporting operation of the system 1100. The infrastructure 1115 may also or instead include location or identification tags or hardware, a power supply for powering modules 1120 or other hardware, communications infrastructure as described herein, a wired intra-garment network, or supplemental components such as a processor, a Global Positioning System (GPS), a timing device, e.g., for synchronizing signals from multiple garments, a beacon for synchronizing signals among multiple modules 1120, and so forth. More generally, any hardware, software, or combination of these suitable for augmenting operation of the garment 1110 and a physiological monitoring system using the garment 1110 may be incorporated as infrastructure 1115 into the garment 1110 as contemplated herein.

The modules 1120 may generally be sized and shaped for placement on or within the one or more designated areas 1112 of the garment 1110. For example, in certain implementations, one or more of the modules 1120 may be permanently affixed on or within the garment 1110. In such instances, the modules 1120 may be washable. Also or instead, in certain implementations, one or more of the modules 1120 may be removable and replaceable relative to the garment 1110. In such instances, the modules 1120 need not be washable, although a module 1120 may be designed to be washable and/or otherwise durable enough to withstand a prolonged period of engagement with a designated area 1112 of the garment 1110. A module 1120 may be capable of being positioned in more than one of the designated areas 1112 of the garment 1110. That is, one or more of the plurality of modules 1120 may be configured to sense data using a physiological sensor 1122 in a plurality of designated areas 1112 of the garment 1110.

Removable and replaceable modules 1120 may provide several advantages such as ease of garment care (e.g., washing) and power management (e.g., removal for recharging). Furthermore, removability may facilitate replacement and/or repositioning of modules within the garment 1110 for different sensing activities or other reconfigurations, replacement of damaged or defective modules 1120, and so forth.

A module 1120 may include one or more physiological sensors 1122 and a communications interface 1124 programmed to transmit data from at least one of the physiological sensors 1122. For example, the physiological sensors 1122 may include one or more of a heart rate monitor, an oxygen monitor (e.g., a pulse oximeter), a thermometer, an accelerometer, a gyroscope, a position sensor, a Global Positioning System, a clock, a galvanic skin response (GSR) sensor, or any other electrical, acoustic, optical, or other sensor or combination of sensors and the like useful for physiological monitoring, environmental monitoring, or other monitoring as described herein. In one aspect, the physiological sensors 1122 may include a conductivity sensor or the like used for electromyography, electrocardiogramalectroencephalography, or other physiological sensing based on electrical signals. The data received from the physiological sensors 1122 may include at least one of heart rate data, muscle oxygen saturation data, temperature data, movement data, position/location data, environmental data, temporal data, and so on.

In one aspect, a module 1120 may be configured for use on multiple body locations. For example, the module 1120 may be one of the wrist-worn sensors described above. The module 1120 may be adapted for use with a garment 1110 in various ways. In one aspect, the module 1120 may have relatively smooth, continuous exterior surfaces to facilitate sliding into and out of a pocket, such as any of the pockets described herein, or any other suitable retaining structure(s). In another aspect, an LED and/or sensor region may protrude from a surface of the module 1120 sufficiently to extend beyond a restraining garment material and into a contact surface of a user. The module 1120 may also include hardware to facilitate such uses. For example, a module 1120 may usefully incorporate a contact sensor for detecting contact with a user. However, the exposed contact surfaces of the module 1120 may be different when retained by a wrist strap (or other limb strap) than when retained by a garment pocket. To facilitate multiple retaining modes, the module 1120 may usefully incorporate two or more contact sensors (such as capacitive sensors or other touch sensors, switches, or the like) at two different locations, each positioned to detect contact with a wearer in a different retaining mode. For example, a module 1120 may include a capacitive sensor adjacent to an optical sensing system that contacts the user's skin when the module 1120 is retained with a wrist strap. The module 1120 may also or instead optically detect contact when the capacitive sensor is covered by a garment fabric or the like that prevents direct skin contact, or a second capacitive sensor may be placed within another region exposed by the garment 1110 retaining system. In another aspect, the garment 1110 may include a capacitive sensor that provides a signal to the module 1120, or to some other system controller or the like, when a region of the garment near the module 1120 is in contact with a user's skin.

In one aspect, the physiological sensors 1122 may include a heart rate monitor or pulse sensor, e.g., where heart rate is optically detected from an artery, such as the radial artery. In one embodiment, the garment 1110 may be configured such that a module 1120 is positioned on a user's wrist, where a physiological sensor 1122 of the module 1120 is secured over the user's radial artery or other blood vessel. Secure connection and placement of a pulse sensor over the radial artery or other blood vessel facilitates measurement of heart rate, pulse oxygen, and the like. It will be understood that this configuration is provided by way of example only, and that other sensors, sensor positions, and monitoring techniques may also or instead be employed without departing from the scope of this disclosure.

In some embodiments, heart rate data may be acquired using an optical sensor coupled with one or more light emitting diodes (LEDs), all in contact with the user 1101. To facilitate optical sensing, the garment 1110 may be designed to maintain a physiological sensor 1122 in secure, continual contact with the skin, and reduce interference of outside light with optical sensing by the physiological sensor 1122.

Thus, certain embodiments include one or more physiological sensors 1122 configured to provide continuous measurements of heart rate using photoplethysmography or the like. The physiological sensor 1122 may include one or more light emitters for emitting light at one or more desired frequencies toward the user's skin, and one or more light detectors for received light reflected from the user's skin. The light detectors may include a photo-resistor, a phototransistor, a photodiode, and the like. A processor may process optical data from the light detector(s) to calculate a heart rate based on the measured, reflected light. The optical data may be combined with data from one or more motion sensors, e.g., accelerometers and/or gyroscopes, to minimize or eliminate noise in the heart rate signal caused by motion or other artifacts. The physiological sensor 1122 may also or instead provide at least one of continuous motion detection, environmental temperature sensing, electrodermal activity (EDA) sensing, galvanic skin response (GSR) sensing, and the like.

The system 1100 may include different types of modules 1120. For example, a number of different modules 1120 may each provide a particular function. Thus, the garment 1110 may house one or more of a temperature module, a heart rate/PPG module, a muscle oxygen saturation module, a haptic module, a wireless communication module, or combinations thereof, any of which may be integrated into a single module 1120 or deployed in separate modules 1120 that can communicate with one another. Some measurements such as temperature, motion, optical heart rate detection, and the like, may have preferred or fixed locations, and pockets or fixtures within the garment 1110 may be adapted to receive specific types of modules 1120 at specific locations within the garment 1110. For example, motion may preferentially be detected at or near extremities while heart rate data may preferentially be gathered near major arteries. In another aspect, some measurements such as temperature may be measured anywhere, but may preferably be measured at a single location in order to avoid certain calibration issues that might otherwise arise through arbitrary placement.

In another aspect, the system 1100 may include two or more modules 1120 placed at different locations and configured to perform differential signal analysis. For example, the rate of pulse travel and the degree of attenuation in a cardiac signal may be detected using two or more modules at two or more locations, e.g., at the bicep and wrist of a user, or at other locations similarly positioned along an artery. These multiple measurements support a differential analysis that permits useful inferences about heart strength, pliability of circulatory pathways, and other aspects of the cardiovascular system that may indicate cardiac age, cardiac health, cardiac conditions, and so forth. Similarly, muscle activity detection might be measured at different locations to facilitate a differential analysis for identifying activity types, determining muscular fitness, and so forth. More generally, multiple sensors can facilitate differential analysis. To facilitate this type of analysis with greater precision, the garment infrastructure may include a beacon or clock for synchronizing signals among multiple modules, particularly where data is temporarily stored locally at each module, or where the data is transmitted to a processor from different locations wirelessly where packet loss, latency, and the like may present challenges to real time processing.

The communications interface 1124 may be any as described herein, for example including any of the features of the network interface 1104 described above. The communications interface 1124 may be a separate device that provides the ability for the modules 1120 to communicate with one another and/or with other components of the system 1100), or there may be a central module that communicates with other modules 1120 (or with another component of the system 1100). It will be understood that communications may usefully be secured using any suitable encryption technology in order to ensure privacy and security of user data. This may, for example, include encryption for local (wired or wireless) communications among the modules 1120 and/or controller 1130 within the garment 1110. This may also or instead include encryption for remote communications to a server and other remote resources. In one aspect, the garment 1110 and/or controller 1130 may provide a cryptographic infrastructure for securing local communications, e.g., by managing public/private key pairs for use in asymmetric encryption, authentication, digital signatures, and so forth. The keys for this infrastructure may also or instead be managed by an external, trusted third party.

The controller 1130 may be configured, e.g., by computer executable code or the like, to determine a location of the module 1120. This may be based on contextual measurements such as accelerometer data from the module 1120, which may be analyzed by a machine learning model or the like to infer a body position. In another aspect, this may be based on other signals from the module 1120. For example, signals from sensors such as photodiodes, temperature sensors, resistors, capacitors, and the like may be used alone or in combination to infer a body position. In another aspect, the location may be determined based on a proximity of a module 1120 to a proximity sensor, RFID tag, or the like at or near one of the designated areas 1112 of the garment 1110. Based on the location, the controller 1130 may adapt operation of the module 1120 for location-specific operation. This may include selecting filters, processing models, physiological signal detections, and the like. It will be understood that operations of the controller 1130, which may be any controller, microcontroller, microprocessor, or other processing circuitry, or the like, may be performed in cooperation with another component of the system 1100 such as the processor 1140 described herein, one or more of the modules 1120, or another computing device. It will also be understood that the controller 1130 may be located on a local component of the system 1100 (e.g., on the garment 1110, in a module 1120, and so on) or as part of a remote processing facility 1150, or some combination of these. Thus, in an aspect, a controller 1130 is included in at least one of the plurality of modules 1120. And, in another aspect, the controller 1130 is a separate component of the garment 1110, and serves to integrate functions of the various modules 1120 connected thereto. The controller 1130 may also or instead be remote relative to each of the plurality of modules 1120, or some combination of these.

Location detection (i.e., of the modules 1120 and/or physiological sensors 1122) may also usefully be recorded and used in a number of ways by a human user and/or by the system 1100. For example, a detected location may be stored, along with the corresponding garment, so that a user can retrieve a placement history and replace the module 1120 to a previous location for a particular garment as desired. In another aspect, the detected location may be used by the system 1100 to analyze data and make garment specific recommendations. For example, the system 1100 may evaluate the quality of a signal, e.g., using any conventional metrics such as signal-to-noise ratio, or using quality metrics more specific to physiological signals such as correlation to an expected signal or pulse shape, consistency with a rate or magnitude typical for a sensor, pulse-to-pulse consistency for a particular user, or any other measure of signal quality using statics, machine learning, digital signal processing techniques, or the like. A quality metric, however derived, may be used in turn to recommend specific placements of a module 1120 on a garment 1110 for a user, or to recommend a particular garment 1110 for the user. Thus, for example, after acquiring data over a range of garments and activities, the system 1100 may generate a user-actionable recommendation such as, “It appears that when you are jogging, the most accurate heart rate signals can be obtained when you are wearing an XL shirt model number xxxxxx. You may wish to wear this shirt for active workouts, and you may wish to purchase more of this type of shirt for regular use.” As another example, the user-actionable recommendation may suggest: “It appears that one of your modules is not obtaining accurate temperature readings when located on your sleeve elastic band. You may wish to try a different location for this module, or to try a different garment.” More generally, data quality may be measured for a number of different modules at different locations in different garments during different activities, and this data may be used to generate customized recommendations for a user on a per-garment and per-location basis. These recommendations may also be tailored to specific activity types where this data is accurately recorded by the system 1100, either from user input, automatic detection, or some combination of these.

The controller 1130 may be configured to control one or more of (i) sensing performed by a physiological sensor 1122 of the module 1120 and (ii) processing by the module 1120 of the data received from a physiological sensor 1122. That is, in certain aspects, the combination of sensors in the module 1120 may vary based on where it is intended to be located on a garment 1110. In another aspect, processing of data from a module 1120 may vary based on where it is located on a garment 1110. In this latter aspect, a processing resource such as the controller 1130 or some other local or remote processing resource coupled to the module 1120 may detect the location and adapt processing of data from the module 1120 based on the location. This may, for example, include a selection of different models, algorithms, or parameters for processing sensed data.

In another aspect, this may include selecting from among a variety of different activity recognition models based on the detected location. For example, a variety of different activity recognition models may be developed such as machine learning models, lookup tables, analytical models, or the like, which may be applied to accelerometer data to detect an activity type. Other motion data such as gyroscope data may also or instead be used, and activity recognition processes may also be augmented by other potentially relevant data such as data from a barometer, magnetometer, GPS system, and so forth. This may generally discriminate, e.g., between being asleep, at rest, or in motion, or this may discriminate more finely among different types of athletic activity such as walking, running, biking, swimming, playing tennis, playing squash, and so forth. While useful models may be developed for detecting activities in this manner, the nature of the detection will depend upon where the accelerometers are located on a body. Thus, a processing resource may usefully identify location first using location detection systems (such as tags, electromechanical bus connections, etc.) built into the garment 1110, and then use this detected location to select a suitable model for activity recognition. This technique may similarly be applied to calibration models, physiological signals processing models, and the like, or to otherwise adapt processing of signals from a module 1120 based on the location of the module 1120.

Determining the location of a module 1120 may include receiving a sensed location for the module 1120. The sensed location may be provided by a proximity detection circuit such as a near-field-communication (NFC) tag, an (active or passive) RFID tag, a capacitance sensor, a magnetic sensor, an electrical contact, a mechanical contact, and the like. Any corresponding hardware for such proximity detections may be disposed on the module 1120 and the garment 1110 for communication therebetween to detect location when appropriate. For example, in one aspect, an NFC tag may be disposed on or within the garment 1110, and the module may include an NFC tag sensor that can detect the tag and read any location-specific information therefrom. Proximity detection may also or instead be performed using capacitively detected contact, electromagnetically detected proximity, mechanical contact, electrical coupling, and the like. In this manner, a garment 1110 may provide information to an installed module 1120 to inform the module 1120, among other things, where the module 1120 is located, or vice versa.

Thus, communication between a module 1120 and the garment 1110 (or a processor of the garment 1110) may be used to determine the location of a module 1120 on the garment 1110. Communication of location information may be enabled using active techniques, passive techniques, or a combination thereof. For example, a thin, flexible, cheap, washable NFC tag may be sewn into the garment 1110 in various locations where a module 1120 may be placed. When a module 1120 is placed in the garment 1110, the module 1120 may query an adjacent NFC tag to determine its location. Furthermore, the NFC technique or other similar techniques may provide other information to the module 1120, including details about the garment 1110 such as the size, whether it is a gender specific piece, the manufacturer information, model or serial number of the garment, stock keeping unit (SKU), and more. Similarly, the tag may encode a unique identifier for the garment 1110 that can be used to obtain other relevant information using an online resource. The module 1120 may also or instead advertise information about itself to the garment 1110 so that the garment 1110 can synchronize processing with other modules 1120, synchronize communication among modules 1120, control or condition signals from the module 1120, and so forth. The module 1120 can then configure itself within the context of the current garment 1110 and associated modules 1120, and/or to perform certain types of monitoring or data processing.

Determining the location of a module 1120 may also or instead be based, at least in part, on an interpretation of the data received from a physiological sensor 1122 of the module 1120. By way of example, movement of a module 1120 as detected by a sensor may provide information that can be used to predict a position on or within the garment 1110. Also or instead, the type of data that is being received from a module 1120 may indicate where the module 1120 is located on the garment 1110. For example, locations may produce unique signatures of acceleration, gyroscope activity, capacitive data, optical data, temperature data, and the like, depending on where the module 1120 is located, and this data may be fused and analyzed in any suitable manner to obtain a location prediction.

According to the foregoing, determining the location of a module 1120 may also or instead include receiving explicit input from the user 1101, which may identify one of the designated areas on the garment 1110, or a general area of the body (e.g., left wrist, right ankle, and so forth). Because the location of the module 1120 relative to the garment 1110 may be determined from an analysis of a plurality of data sources, the system 1100 may include a component (e.g., the processor 1140) that is configured to reconcile one or more potential sources of location of information based on expected reliability, measured quality of data, express user input, and so forth. A prediction confidence may also usefully be generated in this context, which may be used, for example, to determine whether a user should be queried for more specific location information. More generally, any of the foregoing techniques may be used along or in combination, along with a failsafe measure the requests user input when location cannot confidently be predicted. Also or instead, a user may explicitly specify a prediction preemptively, or as an override to an automatically generated prediction.

Once determined using any of the techniques above, the location of a module 1120 may be transmitted for storage and analysis to a remote processing facility 1150, a database 1160, or the like. That is, in addition to the module 1120 using this information locally to configure itself for the location in which it is worn, the module 1120 may communicate this information to other modules 1120, peripherals, or the cloud. Processing this information in the cloud may help an organization determine if a module 1120 has ever been installed on a garment 1110, which locations are most used, and how modules 1120 perform differently in different locations. These analytics may be useful for many purposes, and may, for example, be used to improve the design or use of modules 1120 and garments 1110, either for a population, for a user type, or for a particular user.

As stated above, the system 1100 may further include a processor 1140 and a memory 1142. In general, the memory 1142 may bear computer executable code configured to be executed by the processor 1140 to perform processing of the data received from one or more modules 1120. One or more of the processor 1140 and the memory 1142 may be located on a local component of the system 1100 (e.g., the garment 1110, a module 1120, the controller 1130, and the like) or as part of a remote processing facility 1150 or the like as shown in the figure. Thus, in an aspect, one or more of the processor 1140 and the memory 1142 is included on at least one of the plurality of modules 1120. In this manner, processing may be performed on a central module, or on each module 1120 independently. In another aspect, one or more of the processor 1140 and the memory 1142 is remote relative to each of the plurality of modules 1120. For example, processing may be performed on a connected peripheral device such as smart phone, laptop, local computer, or cloud resource.

The memory 1142 may store one or more algorithms, models, and supporting data (e.g., parameters, calibration results, user selections, and so forth) and the like for transforming data received from a physiological sensor 1122 of the module 1120. In this manner, suitable models, algorithms, tuning parameters, and the like may be selected for use in transforming the data based on the location of the module 1120 as determined by the controller 1130 and/or processor 1140 as described herein. By way of example, algorithms that convert data from an accelerometer in a module 1120 into a count of a user's steps may be different depending on whether the module 1120 is worn on the user's wrist or on the user's waist band. Similarly, the intensity of an LED and corresponding sensitivity of a photodetector may be different for a PPG device placed on the wrist or the thigh. Thus, the module 1120 may self-configure for a location by controlling one or more of sensor types, sensor parameters, processing models, and so forth based on a detected location for the module 1120.

Selection of an algorithm may also or instead include an analysis of one or more of the sensor data, metadata, and the like. By way of example, an algorithm may be selected at least in part based on metadata received from one of the module 1120 and the garment 1110. This metadata may be derived from communication between the module 1120 and the garment 1110—e.g., between a tag and tag reader for exchanging information therebetween. For example, the garment 1110 may include, e.g., stored in a tag such as an NFC tag or other wirelessly readable data source, garment-specific metadata that is readable by or otherwise transmittable to one or more of the plurality of modules 1120, the controller 1130, and the processor 1140. Such garment-specific metadata may include at least one of a type of garment 1110, a size of the garment 1110, garment dimensions, a gender configuration of the garment 1110, a manufacturer, a model number, a serial number, a SKU, a material, fit information, and so on. In one aspect, this information may be provided with one or more of the location identification tags described herein. In another aspect, the garment 1110 may include an additional tag at a suitable location (e.g., near or accessible to a processor or controller) that provides garment-specific information while other tags provide location-specific information.

The metadata may also or instead include at least one of a gender of the user 1101, a weight of the user 1101, a height of the user 1101, an age of the user 1101, metadata associated with the garment 1110 (e.g., the garment size, type, material, etc.), and the like. The metadata may be derived, at least in part, from user-provided input, or otherwise from information derived from the user 1101 such as a user's account information as a participant in the system 1100. By way of example, a processing algorithm may be selected depending on the material of the garment 1110 as communicated by its serial or model number in an identification tag, the physiology of the user 1101 as implied by the garment size, and so on. The metadata may also or instead be used to verify the authenticity of the garment 1110 and otherwise control access to the garment 1110 and/or modules 1120 coupled to the garment 1110. In one aspect, metadata (e.g., size, material) may be encoded directly into the garment metadata. In another aspect, the garment 1110 may publish a unique identifier that can be used to retrieve related information from a manufacturer or other data source. This latter approach advantageously permits correlation of garment-specific data with other user-specific data such as height, weight, body composition, and so forth.

Simply knowing a priori where a module 1120 is positioned may allow for the use of algorithms that have been developed to perform optimally in that particular location. This can relieve a significant computational burden otherwise borne by the module 1120 to analytically evaluate location based on available signals. Other information may also or instead be used to select an optimal algorithm. For example, based on the gender or dimensions of a garment, the algorithm may employ different models or different model parameters.

The processor 1140 may be configured to assess the quality of the data received from a physiological sensor 1122 of the module 1120. For example, the processor 1140 may be configured to provide, based on the quality of the data, a recommendation regarding at least one of the location of a module 1120 and an aspect of the garment 1110 (e.g., size, fit, material, and so on). For example, the processor 1140 may be configured to detect when the garment does not properly fit the wearer for acquisition of physiological data, for example, by detecting when a module is moving (e.g., from accelerometer data) but data quality is poor or absent for a sensed physiological signal. In general, the garment 1110 may store its own identifier and/or metadata, e.g., as described herein, or garment identification data may be stored in tags, e.g., at designated areas 1112 of the garment 1110. The processor 1140 may be configured to use this garment identification information and/or metadata to provide a recommendation regarding a different garment 1110 for the user 1101, or for an adjustment to the current garment 1110. For example, if a particular garment 1110 seems to result in low-quality data, the user 1101 could be encouraged to select an alternative size, or to make some other adjustment. Moreover, data on how many times a garment 1110 is used may be gathered and used to inform business decisions, for example, which garments 1110 provide the highest-quality data, and which garments 1110 are most preferred by users 1101.

The system 1100 may further include a database 1160, which may be located remotely and in communication with the system 1100 via the data network 1102. The database 1160 may store data related to the system 1100 such as any discussed herein—e.g., sensed data, processed data, transformed data, metadata, physiological signal processing models and algorithms, personal activity history, and the like. The system 1100 may further include one or more servers 1170 that host data, provide a user interface, process data, and so forth in order to facilitate use of the modules 1120 and garments 1110 as described herein.

It will be appreciated that the garment 1110, modules 1120, and accompanying garment infrastructure and remote networking/processing resources, may advantageously be used in combination to improve physiological monitoring and achieve modes of monitoring not previously available.

One or more of the devices and systems described herein may include circuitry for both wireless charging and wireless data transmission, e.g., where the corresponding circuits can operate independently from one another, and where the corresponding antennae are located proximal to one another (for instance, the circuitry for wireless charging and the circuitry for wireless data transmission may include separate coils disposed substantially along the same plane, or otherwise in relative close proximity in a device or system). In such aspects, one or more measures may be taken so that a wireless data transfer process does not interfere with a wireless power transfer process, more specifically by coupling the data circuitry into the electromagnetic field for the wireless power transfer in a manner that alters the resonant frequency or otherwise destructively interferes with power transfer, thereby decreasing efficiency when charging a device. For example, a switch may be included to disable circuitry for data transmission when certain wireless charging activity is present, thereby allowing for relatively unimpeded and efficient wireless charging of a device. The switch may also be operable to enable operation of data transmission circuitry when certain wireless charging activity is not present.

Thus, for example, in the context of a physiological monitor, such as any of those described herein, the physiological monitor may include both a wireless power receiver (or similar) and a wireless data tag reader (or similar). In general, these sub-systems may conform to one or more Near Field Communication (NFC) specifications for protocols and physical architectures, or any other standards suitable for wireless power and data transmission. The power circuitry may be used, e.g., to charge a battery on the physiological monitor so that the device can be recharged without physically connecting to a power source. The data circuitry may be used, e.g., as a wireless data tag reader or the like to read data from nearby data sources such as identification tags in user apparel and the like. In general, the physiological monitor may include separate circuitry (separate coils) for these wireless power and data systems, such as separate processing circuitry and/or separate antennae. The antennae may be disposed substantially along the same plane of the physiological monitor (e.g., with one coil disposed substantially inside or adjacent to the other). In one aspect, the antennae may be in parallel planes, however, it will be noted that distance tolerances for NFC standard devices are relatively small, and the physical housing for these antennae will preferably enforce an identical or substantially identical distance for both antennae in such architectures. In this context, the positions of the antennae may be as close to parallel as possible within reasonable manufacturing tolerances, or as close to parallel as possible when disposed on two different layers of a shared printed circuit board, or preferably, when disposed on a single layer of a shared printed circuit board. The physiological monitor may further include a switch (e.g., a radio frequency (RF) switch or the like) in-line with the coil for the wireless data tag reader to disable the wireless data tag reader when power is being received to mitigate any effects on the efficiency of the wireless power transfer process. In particular, the switch may be configured to open when power is being received and may be configured to close when the physiological monitor is looking for data tag to read.

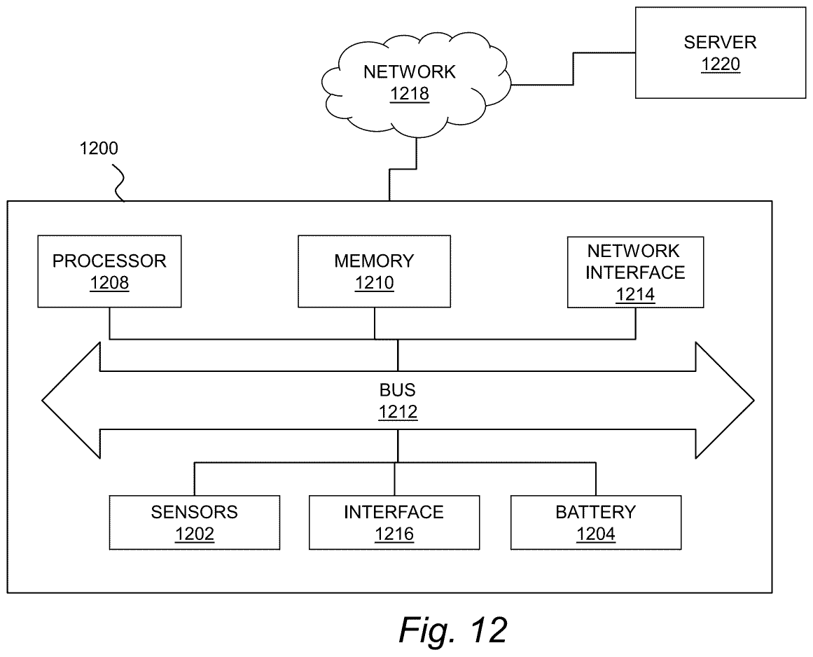

FIG. 12 is a block diagram of a computing device 1200. The computing device 1200 may, for example, be a device used for continuous physiological monitoring, or any other device supporting a physiological monitor in the systems and methods described herein. The device may also or instead be any of the local computing devices described herein, such as a desktop computer, laptop computer, smart phone. The device may also or instead be any of the remote computing resources described herein, such as a web server, a cloud database, a file server, an application server, or any other remote resource or the like. While described as a physical device, it will be understood that the exemplary computing device 1200 may also or instead be realized as a virtual computing device such as a virtual machine executing a web server or other remote resource in a cloud computing platform. In general, the device 1200 may include one or more sensors 1202, a battery 1204, a processor 1208, a memory 1210, a network interface 1214, and a user interface 1216, or virtual instances of one or more of the foregoing, interconnected by a bus 1212 for transmission of data and power therebetween.

The sensors 1202 may include any sensor or combination of sensors suitable for heart rate monitoring as contemplated herein, as well as sensors 1202 for detecting calorie burn, position (e.g., through a Global Positioning System or the like), motion, activity and so forth. In one aspect, this may include optical sensing systems including LEDs or other light sources, along with photodiodes or other light sensors, which can be used in combination for photoplethysmography measurements of heart rate, pulse oximetry measurements, and other physiological monitoring.

The sensors 1202 may also or instead include one or more sensors for activity measurement. In some embodiments, the system may include one or more multi-axes accelerometers and/or gyroscope to provide a measurement of activity. In some embodiments, the accelerometer may further be used to filter a signal from the optical sensor for measuring heart rate and to provide a more accurate measurement of the heart rate. In some embodiments, the wearable system may include a multi-axis accelerometer to measure motion and calculate distance. Motion sensors may be used, for example, to classify or categorize activity, such as walking, running, performing another sport, standing, sitting or lying down. The sensors 1202 may, for example, include a thermometer for monitoring the user's body or skin temperature. In one embodiment, the sensors 1202 may be used to recognize sleep based on a temperature drop, Galvanic Skin Response data, lack of movement or activity according to data collected by the accelerometer, reduced heart rate as measured by the heart rate monitor, and so forth. The body temperature, in conjunction with heart rate monitoring and motion, may be used, e.g., to interpret whether a user is sleeping or just resting, as well as how well an individual is sleeping. The body temperature, motion, and other sensed data may also be used to determine whether the user is exercising, and to categorize and/or analyze activities as described in greater detail below. In another aspect, the sensors 1202 may include one or more contact sensors, such as a capacitive touch sensor or resistive touch sensor, for detecting placement of a physiological monitor for use on a user. More generally, the sensors 1202 may include any sensor or combination of sensors suitable for monitoring geographic location, physiological state, exertion, movement, and so forth in any manner useful for physiological monitoring as contemplated herein.

The battery 1204 may include one or more batteries configured to allow continuous wear and usage of the wearable system. In one embodiment, the wearable system may include two or more batteries, such as a removable battery that may be removed and recharged using a charger, along with an integral battery that maintains operation of the device 1200 while the main battery charges. In another aspect, the battery 1204 may include a wireless rechargeable battery that can be recharged using a short range or long range wireless recharging system.

The processor 1208 may include any microprocessor, microcontroller, signal processor or other processor or combination of processors and other processing circuitry suitable for performing the processing steps described herein. In general, the processor 1208 may be configured by computer executable code stored in the memory 1210 to provide activity recognition and other physiological monitoring functions described herein.

In general the memory 1210 may include one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, optical disks, USB flash drives), and the like. In one aspect, the memory 1210 may include a computer system memory or random access memory, such as DRAM, SRAM, EDO RAM, and the like. The memory 1210 may include other types of memory as well, or combinations thereof, as well as virtual instances of memory, e.g., where the device is a virtual device. In general, the memory 1210 may store computer readable and computer-executable instructions or software for implementing methods and systems described herein. The memory 1210 may also or instead store physiological data, user data, or other data useful for operation of a physiological monitor or other device described herein, such as data collected by sensors 1202 during operation of the device 1200.

The network interface 1214 may be configured to wirelessly communicate data to a server 1220, e.g., through an external network 1218 such as any public network, private network, or other data network described herein, or any combination of the foregoing including, e.g., local area networks, the Internet, cellular data networks, and so forth. Where the device is a physiological monitoring device, the network interface 1214 may be used, e.g., to transmit raw or processed sensor data stored on the device 1200 to the server 1220, as well as to receive updates, receive configuration information, and otherwise communicate with remote resources and the user to support operation of the device. More generally, the network interface 1214 may include any interface configured to connect with one or more networks, for example, a Local Area Network (LAN), a Wide Area Network (WAN), the Internet, or a cellular data network through a variety of connections including, but not limited to, standard telephone lines, LAN or WAN links (for example, 802.11, T1, T3, 56 kb, X.25), broadband connections (for example, ISDN, Frame Relay, ATM), wireless connections, or some combination of any or all of the above. The network interface 1214 may include a built-in network adapter, network interface card, PCMCIA network card, card bus network adapter, wireless network adapter, USB network adapter, modem or any other device suitable for interfacing the computing device 1200 to any type of network capable of communication and performing the operations described herein.

The user interface 1216 may include any components suitable for supporting interaction with a user. This may, for example, include a keypad, display, buzzer, speaker, light emitting diodes, and any other components for receiving input from, or providing output to, a user. In one aspect, the device 1200 may be configured to receive tactile input, such as by responding to sequences of taps on a surface of the device to change operating states, display information and so forth. The user interface 1216 may also or instead include a graphical user interface rendered on a display for graphical user interaction with programs executing on the processor 1208 and other content rendered by a physical display of device 1200.

All documents mentioned herein are hereby incorporated by reference in their entirety. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context. Thus, the term “or” should generally be understood to mean “and/or” and so forth.

Recitation of ranges of values herein are not intended to be limiting, referring instead individually to any and all values falling within the range, unless otherwise indicated herein, and each separate value within such a range is incorporated into the specification as if it were individually recited herein. The words “about,” “approximately” or the like, when accompanying a numerical value, are to be construed as indicating a deviation as would be appreciated by one of ordinary skill in the art to operate satisfactorily for an intended purpose. Similarly, words of approximation such as “approximately” or “substantially” when used in reference to physical characteristics, should be understood to contemplate a range of deviations that would be appreciated by one of ordinary skill in the art to operate satisfactorily for a corresponding use, function, purpose, or the like. Ranges of values and/or numeric values are provided herein as examples only, and do not constitute a limitation on the scope of the described embodiments. Where ranges of values are provided, they are also intended to include each value within the range as if set forth individually, unless expressly stated to the contrary. The use of any and all examples, or exemplary language (“e.g.,” “such as,” or the like) provided herein, is intended merely to better describe the embodiments and does not pose a limitation on the scope of the embodiments. No language in the specification should be construed as indicating any unclaimed element as essential to the practice of the embodiments.

In the following description, it is understood that terms such as “first,” “second,” “top,” “bottom,” “up,” “down,” “above,” “below,” and the like, are words of convenience and are not to be construed as limiting terms unless specifically stated to the contrary.

The term “continuous,” as used herein in connection with heart rate data, refers to the acquisition of heart rate data at a sufficient frequency to enable detection of individual heartbeats, and also refers to the collection of heart rate data over extended periods such as a day or more (including acquisition throughout the day and night). More generally with respect to physiological signals that might be monitored by a wearable device, “continuous” or “continuously” will be understood to mean continuously at a rate and duration suitable for the intended time-based processing, and physically at an inter-periodic rate (e.g., multiple times per heart beat, respiration, and so forth) sufficient for resolving the desired physiological characteristics such as heart rate, heart rate variability, heart rate peak detection, pulse shape, and so forth. At the same time, continuous monitoring is not intended to exclude ordinary data acquisition interruptions such as temporary displacement of monitoring hardware due to sudden movements, changes in external lighting, loss of electrical power, physical manipulation or adjustment by a wearer, physical displacement of monitoring hardware due to external forces, and so forth. It will also be noted that heart rate data or a monitored heart rate, in this context, may more generally refer to raw sensor data, or processed data therefrom such as heart rate data, signal peak data, heart rate variability data, or any other physiological or digital signal suitable for recovering heart rate information as contemplated herein, and that heart rate data may generally be captured over some historical period that can be subsequently correlated to various metrics such as sleep states, activity recognition, resting heart rate, maximum heart rate, and so forth.

The term “computer-readable medium,” as used herein, refers to a non-transitory storage hardware, non-transitory storage device or non-transitory computer system memory that may be accessed by a controller, a microcontroller, a microprocessor, a computational system, or a module of a computational system to encode thereon computer-executable instructions or software programs. The “computer-readable medium” may be accessed by a computational system or a module of a computational system to retrieve and/or execute the computer-executable instructions or software programs encoded on the medium. The non-transitory computer-readable media may include, but are not limited to, one or more types of hardware memory, non-transitory tangible media (for example, one or more magnetic storage disks, one or more optical disks, one or more USB flash drives), computer system memory or random access memory (such as, DRAM, SRAM, EDO RAM) and the like.

The above systems, devices, methods, processes, and the like may be realized in hardware, software, or any combination of these suitable for the control, data acquisition, and data processing described herein. This includes realization in one or more microprocessors, microcontrollers, embedded microcontrollers, programmable digital signal processors or other programmable devices or processing circuitry, along with internal and/or external memory. This may also, or instead, include one or more application specific integrated circuits, programmable gate arrays, programmable array logic components, or any other device or devices that may be configured to process electronic signals. It will further be appreciated that a realization of the processes or devices described above may include computer-executable code created using a structured programming language such as C, an object oriented programming language such as C++, or any other high-level or low-level programming language (including assembly languages, hardware description languages, and database programming languages and technologies) that may be stored, compiled or interpreted to run on one of the above devices, as well as heterogeneous combinations of processors, processor architectures, or combinations of different hardware and software.

Thus, in one aspect, each method described above, and combinations thereof may be embodied in computer executable code that, when executing on one or more computing devices, performs the steps thereof. In another aspect, the methods may be embodied in systems that perform the steps thereof, and may be distributed across devices in a number of ways, or all of the functionality may be integrated into a dedicated, standalone device or other hardware. The code may be stored in a non-transitory fashion in a computer memory, which may be a memory from which the program executes (such as random access memory associated with a processor), or a storage device such as a disk drive, flash memory or any other optical, electromagnetic, magnetic, infrared, or other device or combination of devices. In another aspect, any of the systems and methods described above may be embodied in any suitable transmission or propagation medium carrying computer-executable code and/or any inputs or outputs from same. In another aspect, means for performing the steps associated with the processes described above may include any of the hardware and/or software described above. All such permutations and combinations are intended to fall within the scope of the present disclosure.

The method steps of the implementations described herein are intended to include any suitable method of causing such method steps to be performed, consistent with the patentability of the following claims, unless a different meaning is expressly provided or otherwise clear from the context. So, for example, performing the step of X includes any suitable method for causing another party such as a remote user, a remote processing resource (e.g., a server or cloud computer) or a machine to perform the step of X. Similarly, performing steps X, Y, and Z may include any method of directing or controlling any combination of such other individuals or resources to perform steps X, Y, and Z to obtain the benefit of such steps. Thus, method steps of the implementations described herein are intended to include any suitable method of causing one or more other parties or entities to perform the steps, consistent with the patentability of the following claims, unless a different meaning is expressly provided or otherwise clear from the context. Such parties or entities need not be under the direction or control of any other party or entity and need not be located within a particular jurisdiction.

It will be appreciated that the methods and systems described above are set forth by way of example and not of limitation. Numerous variations, additions, omissions, and other modifications will be apparent to one of ordinary skill in the art. In addition, the order or presentation of method steps in the description and drawings above is not intended to require this order of performing the recited steps unless a particular order is expressly required or otherwise clear from the context. Thus, while particular embodiments have been shown and described, it will be apparent to those skilled in the art that various changes and modifications in form and details may be made therein without departing from the spirit and scope of this disclosure and are intended to form a part of the invention as defined by the following claims.

The above illustrative examples of various aspects and implementations refer to prediction models or machine learning models. The skilled person will appreciate that, even if not expressly stated, a prediction model or machine learning model can be trained using standard training approaches as known in the art. For example, a standard approach for training a prediction model comprises obtaining relevant training data (e.g., using known data sources, databases, or data sets) and performing cross-validation to train the prediction model on the training data. Typically, cross-validation involves splitting the training data into K-folds (approximately equal partitions or sets of the training data) and withholding a single fold as a test set and, one by one, using one of the remaining folds as a validation set and the remaining K-2 folds as a training set. The model is then repeatedly trained on the training set using different model hyperparameters and the performance validated on the validation set. Once the best performing hyperparameters are obtained, the model trained according to the best hyperparameters are evaluated on the test set. For training classification models, the cross-validation strategy can be stratified such that the proportion of training instances within each category or class is approximately the same across each fold. Model hyperparameters can be selected using any suitable approach such as grid search or randomized search. Model performance can be estimated using any suitable performance measure and is dependent on the type of model being trained (e.g., mean square error for regression, binary cross entropy for classification, ranking loss for ranking, etc.).

1-38. (canceled)

39. A method comprising:
providing a machine learning model trained to provide an estimated respiratory waveform based on one or more features of heart pulse samples;
receiving, from a wearable physiological monitor worn by a user, pulse data including a plurality of heart pulse samples of the user during a time window;
receiving accelerometer data from the wearable physiological monitor during the time window;
extracting the one or more features from the pulse data;
obtaining the estimated respiratory waveform for the user during the time window with the machine learning model based on the extracted features; and
calculating a plurality of respiratory onsets for the user during the time window based on a plurality of local extrema of the estimated respiratory waveform for the user and local extrema determined from the accelerometer data obtained from the wearable physiological monitor during the time window.

40. The method of claim 39, wherein the wearable physiological monitor includes a photoplethysmography monitor.

41. A computer program product comprising executable code embodied in a non-transitory computer readable medium that, when executing on one or more computing devices, performs the steps of:
receiving, from a wearable physiological monitor worn by a user, pulse data including a plurality of heart pulse samples of the user during a time window;
mapping the pulse data into a feature space, wherein the feature space encodes individual heart pulse samples according to respiratory cycle stage;
generating a first estimated respiratory waveform of the user during the time window from the pulse data mapped to the feature space; and
calculating a plurality of respiratory onsets for the user during the time window based on a plurality of local extrema of the first estimated respiratory waveform.

42. The computer program product of claim 41, wherein the executable code, when executing on one or more computing devices, further performs the step of calculating a respiratory rate variability score based on a metric calculated using the plurality of respiratory onsets.

43. The computer program product of claim 42, wherein the executable code, when executing on one or more computing devices, further performs the step of determining a physiological condition associated with the user based on the respiratory rate variability score.

44. The computer program product of claim 43, wherein the physiological condition is a recovery state of the user.

45. The computer program product of claim 43, wherein the physiological condition is a sleep stage of the user.

46. The computer program product of claim 43, wherein the metric comprises one of: a standard deviation of respiratory cycle lengths (RCL) metric; a standard deviation of successive differences of RCL metric; a root mean square of successive differences of RCL metric; a coefficient of variation of RCL metric; a median absolute deviation from median RCL metric; or a coefficient of variation based on an absolute deviation from median RCL metric.

47. The computer program product of claim 41, wherein the first estimated respiratory waveform is generated based on a first feature of the feature space, the first feature being related to modulation of heart pulse samples with respect to a respiratory cycle of the user.

48. The computer program product of claim 47, wherein the step of mapping the pulse data into the feature space includes the step of extracting the first feature from the plurality of heart pulse samples of the pulse data.

49. The computer program product of claim 47, wherein the first feature is any one of: a heart pulse frequency variation feature; a heart pulse amplitude variation feature; a steady state baseline wander; or a heart pulse gradient variation feature.

50. The computer program product of claim 47, wherein the executable code, when executing on one or more computing devices, further performs the step of generating a second estimated respiratory waveform of the user during the time window from the pulse data mapped to the feature space, wherein the second estimated respiratory waveform is generated based on a second feature of the feature space, the second feature being related to modulation of heart pulse samples with respect to the respiratory cycle of the user.

51. The computer program product of claim 50, wherein the step of mapping the pulse data into the feature space includes the step of extracting the second feature from the plurality of heart pulse samples of the pulse data.

52. The computer program product of claim 51, wherein the plurality of local extrema used to calculate the plurality of respiratory onsets include local extrema of the first estimated respiratory waveform and local extrema of the second estimated respiratory waveform.

53. The computer program product of claim 52, wherein the executable code, when executing on one or more computing devices, further performs the step of determining a weighting for the local extrema, wherein the plurality of respiratory onsets are calculated according to the weighting.

54. The computer program product of claim 41, wherein the plurality of respiratory onsets are further based on local extrema determined from accelerometer data obtained from the wearable physiological monitor during the time window.

55. A system comprising:
a wearable physiological monitor configured to acquire pulse data from a user during a time window, the pulse data including a plurality of heart pulse samples of the user during the time window; and
one or more processors configured to:
map the pulse data into a feature space, wherein the feature space encodes individual heart pulse samples according to respiratory cycle stage,
generate a first estimated respiratory waveform of the user during the time window from the pulse data mapped to the feature space, and
calculate a plurality of respiratory onsets for the user during the time window based on a plurality of local extrema of the first estimated respiratory waveform.

56. The system of claim 55, wherein the one or more processors include a processor in at least one of the wearable physiological monitor and a remote resource configured to receive data through a data network from the wearable physiological monitor.

57. The system of claim 55, wherein the wearable physiological monitor is configured to acquire accelerometer data during the time window, the plurality of respiratory onsets further based on local extrema of the accelerometer data.

58. The system of claim 55, wherein the time window corresponds to a portion of a sleep session of the user.
